(19) United States
(12) Patent Application Publication
(10) Pub. No.: US 2015/0104446 A1
(43) Pub. Date: Apr. 16, 2015

(54) GENETIC AND ENVIRONMENTAL MARKERS TO IDENTIFY INFANTS AT RISK FOR SEVERE LUNG DISEASE

(71) Applicant: Vanderbilt University, Nashville, TN (US)

(72) Inventors: Fernando P. POLACK, Nashville, TN (US); Steven R. KLEEBERGER, Research Triangle Park, NC (US)

(21) Appl. No.: 14/511,763

(22) Filed: Oct. 10, 2014

Related U.S. Application Data
(60) Provisional application No. 61/889,172, filed on Oct. 10, 2013.
(60) Provisional application No. 62/017,664, filed on Jun. 26, 2014.

(51) Int. Cl. C12Q 1/68; A61K 31/137; A61K 31/56; C07K 16/24; C07K 16/10

(52) U.S. Cl. CPC C12Q 1/6883; C07K 16/247; C07K 16/1027; A61K 31/56; A61K 31/137; A61K 2039/505
USPC 424/133.1; 424/158.1; 424/159.1; 514/171

(57) ABSTRACT

Provided herein are methods and compositions for determining the susceptibility of infants to severe respiratory syncytial virus (RSV) infections. Also provide are methods of treating said subjects prophylactically to reduce the incidence of such infections.

This application claims benefit of priority to U.S. Provisional Application Ser. No. 61/889,172, filed Oct. 10, 2013, and U.S. Provisional Application Ser. No. 62/017,664, filed Jun. 26, 2014, the entire contents of each of which are hereby incorporated by reference.

### BACKGROUND

1. Field

The present disclosure relates generally to the fields of medicine, genetics, and virology. More particularly, it concerns identifying subjects at risk of severe bronchiolitis caused by respiratory syncytial virus (RSV).

2. Description of Related Art

Respiratory syncytial virus (RSV) is the most frequent cause of hospitalization in infants worldwide¹. An estimated 30-70% of infants develop bronchiolitis upon primary RSV infection, 1-3% are hospitalized (Collins et al., 2001, Simoes, 2003 and Ferolla et al., 2013). Even though certain risk factors for hospitalization have been identified (Collins et al., 2001, Simoes, 2003 and Ferolla et al., 2013), the mechanism of severe RSV bronchiolitis is not well understood.

Three hypotheses have been widely accepted as probable explanations for disease severity (Hoffman et al., 2004). The first attributes severe RSV bronchiolitis to direct virus damage of the respiratory tract, and is supported by studies where higher doses of candidate intranasal vaccine viruses increased symptoms in recipients (Karron, 2005). A second hypothesis, based on the small diameter of bronchioles in young infants and high levels of inflammatory cytokines during RSV lower respiratory illness, postulates that severe bronchiolitis is a consequence of inflammation (Hull et al., 2000). The final explanation ascribes severe RSV bronchiolitis to a CD4⁺ T helper type 2 (Th2) polarization of the immune response in the respiratory tract (Openshaw, 1995 and McNamara et al., 2004), and is supported by resemblance of symptoms in bronchiolitis and asthma, high Th2 cytokine levels in severely ill infants, and genetic association studies of Th2 genes (McNamara et al., 2004, Hoebee et al., 2003 and Webb et al., 2003).

Toll-like receptor 4 (TLR4) is associated with all three hypotheses, and its activation has been reported to affect titers in the lungs, inflammation, and Th bias (Kurt-Jones et al., 2000 and Haynes et al., 2001). However, studies of the role of TLR4 in RSV pathogenesis yield conflicting results in mice (Kurt-Jones et al., 2000, Haynes et al., 2001 and Ehl et al., 2004) and in pediatric populations living in different environments (Table 2) (Tal et al., 2004, Inoue et al., 2007, Puthothu et al., 2006, Paulus et al., 2007 and Kresfelder et al., 2011). To the inventors' knowledge, these environmental factors have not been considered in evaluations of RSV pathogenesis. Yet, gene-environment interactions involving TLRs modulate other wheezing illnesses (Smit et al., 2009 and Martinez, 2007).

Two SNPs in TLR4 encode substitutions (Asp299Gly and Thr399Ile) in the ectodomain that are in high linkage disequilibrium, and have been associated with an LPS-hyporesponsive phenotype (Arbour et al., 2000). LPS interacts with molecules that allow activation of TLR4, altering responses to different stimuli (Braun-Fahrlander et al., 2002). However, the particular impact of these SNPs on RSV has not been explored.

### SUMMARY

Thus, in accordance with the present disclosure, there is provided a method of prophylactically treating a subject for respiratory syncytial virus (RSV) to reduce the chance of infection comprising (a) obtaining a genomic sample from a infant human subject; (b) determining the presence or absence of the single nucleotide polymorphism (SNP) designated rs4986790 and/or rs4986791; (c) determining lipopolysaccharide (LPS) exposure of said subject, and (d) treating the subject prophylactically if said subject is heterozygous for the SNP designated rs4986790 and/or rs4986791, and LPS exposure is low. The subject may be 1 to 61 days old, or 1 to 12 months old. The sample may be blood, a buccal cell sample, a nasal aspirate, or urine. Treating may comprise administering an anti-RSV antibody (e.g., palivizumab) to said subject, an anti-IL-4 antibody to said subject, a pro-IFNγ agent to said subject, or combinations thereof. Also, treating may comprise administering low LPS levels to said subject.

The method may further comprise treating said subject exhibiting either SNP with albuterol and/or corticosteroids in the event of an acute episode of bronchiolitis. Determining environmental LPS may comprise (a) performing an LPS assay on a sample from said subject's home, or (b) performing a patient questionnaire that determines socio-economic status. LPS may be determined by immunoassay, mass spectrometry, gas chromatography, or bioassay (e.g., a limulus amebocyte assay). Determining the presence of said SNP may comprise sequencing, RFLP analysis, or primer extension.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present disclosure will become apparent from the following detailed description. It should be understood, however, that the detailed description and the specific examples, while indicating preferred embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-F. Pattern recognition receptors and environmental interactions. (FIG. 1A) LPS concentrations in cradles and bed sheets of infants from low vs. high socioeconomic groups. P<0.01. (FIG. 1B) Interleukin-6 levels in respiratory secretions of RSV-infected infants with two major alleles (black bar) and infants with one TLR4D299G allele (white bar). P=0.0002. Data represent mean±SEM. (FIG. 1C) Time from initiation of symptoms to collection of respiratory secretions in RSV-infected infants with two major alleles (black bars) and infants with one TLR4D299G allele (white bars). P=0.251. Data represent mean±SEM. (FIGS. 1D-E) Association of the TLR4 Asp299Gly heterozygous genotype in RSV-infected infants with mild (white bars) and severe (black bars) bronchiolitis in groups exposed to high and low environmental levels of LPS in 2003-2006 (FIG. 1D; P=0.003 for interaction) and 2010-2013 (FIG. 1E; p=0.002 for interaction). (FIG. 1F) Odds ratios and 95% confidence intervals for disease severity in infants from high and low LPS environments in population 1 (2003-2006), population 2 (2010-2013), and both populations combined (p<0.001 for interaction in combination).

FIGS. 2A-D. RSV lung titers are not associated with severity. (FIG. 2A) RSV titers in respiratory secretions from infants with severe (black box) and mild (white box) bronchiolitis. P=0.659. Data represent mean±SEM. (FIG. 2B) Time from initiation of symptoms to collection of respiratory secretions in RSV-infected infants with severe (black bar) and mild (white bar) bronchiolitis. P=0.990. (FIG. 2C) RSV titer in respiratory secretions of infants exposed to low (black box) and high (white box) environmental levels of LPS. P=0.608. Data represent mean±SEM. (FIG. 2D) RSV titer in respiratory secretions of RSV-infected infants with two major alleles (black box) and infants with one TLR4D299G allele (white box). P=0.399.

FIGS. 3A-I. Inflammation in the lungs is determined by environmental exposures. (FIG. 3A) IL-6, (FIG. 3B) IL-8, (FIG. 3C) TNF-α, and (FIG. 3D) IL-1β levels in respiratory secretions from infants with mild severe (black bars) and (white bars) bronchiolitis. P=NS for all comparisons. Data represent mean±SEM. (FIG. 3E) IL-6 and (FIG. 3F) IL-8 levels in respiratory secretions from infants exposed to low (black bars) and high (white bars) environmental levels of LPS. P for IL-6=0.04; P for IL-8=0.003. Data represent mean±SEM. (FIGS. 3G-I) TLR4, CD14 and TLR2 mRNA expression in respiratory secretions of healthy infants exposed to low (black bar) and high (white bar) environmental levels of LPS. P<0.01 for all comparisons.

FIGS. 4A-J. Th2 bias and RSV bronchiolitis. (FIG. 4A) Percent of RSV-infected infants with severe (black bar) and mild (white bar) RSV bronchiolitis with GATA3/T-bet mRNA ratio >1 in respiratory secretions. P=0.02. (FIG. 4B) Percent of high risk (WT and high LPS+TLR4D299Gand low LPS: black bar) and low risk (WT and low LPS+TLR4D299Gand high LPS: white bar) RSV-infected infants with GATA3/T-bet mRNA ratio >1 in respiratory secretions. P<0.0001. (FIG. 4C) Effect of TLR4-environment interaction on severity of RSV bronchiolitis in unadjusted analysis and after adjusting for GATA3/T-bet ratios >1. (FIG. 4D) IFNγ, (FIG. 4E) IL-4 and (FIG. 4F) IL-4/IFNγ levels in respiratory secretions of infants with severe (black bar) and mild (white bar) RSV bronchiolitis. pvalue for IFNγ=0.004; p for IL-4=0.007; p for IL-4/IFNγ=0.005. Data represent mean±SEM. (FIG. 4G) IL-4/IFNγ levels in respiratory secretions of high (black bar) or low (white bar) risk infants; p=0.009. Data represent mean±SEM. (FIG. 4H) IL-9, (FIG. 4I) IL-13 and (FIG. 4J) IL-5 levels in respiratory secretions of infants with severe (black bar) and mild (white bar) RSV bronchiolitis; p for IL-9=0.86, p for IL-13=0.86 and p for IL-5=0.002. Data represent mean±SEM.

FIGS. 5A-H. Th2 bias promotes RSV disease in mice. AHR in mice exposed to 50 mg aerosolized metacholine (mCh) 5 days after infection with 10⁶ pfu IN of RSV line 19 after high (100 μg god; FIG. 5A) or no (FIG. 5B) exposure to bacterial LPS. P=0.029 (FIG. 5A), p=0.006 (FIG. 5B) for wt vs. Tlr4^{+/-}. No differences were observed between uninfected wt and Tlr4^{+/-} controls mice (not shown). (FIG. 5C) IFNγ, (FIG. 5D) IL-4 and (FIG. 5E) IL-4/IFNγ ratios in lung homogenates of high risk (no LPS exposure for Tlr4^{+/-}+ high LPS exposure for wt) compared to low risk (no LPS for wt+high LPS for Tlr4^{+/-}) mice 5 days after inoculation with RSV line 19; p for IFNγ=0.002, p for IL-4=0.018 and p for IL-4/IFNγ ratios=0.011. (FIGS. 5F-G) AHR in BALB/c (wt), Stat-1^{-/-} (deficient T-bet activation), and Stat-6^{-/-} (deficient GATA3 activation) mice exposed to aerosolized metacholine (mCh) 5 days after infection with 10⁶ pfu of RSV line 19. P for Stat-1^{-/-} vs. wt and Stat-6^{-/-}; p<0.05; Stat-6^{-/-} vs wt in FIG. 5E: p<0.05. Data represent mean±SEM. No differences were observed between uninfected controls (FIG. 5H) AHR in RSV-infected, tamoxifen-treated C57BL/6 wt and mice conditionally deficient on GATA3 (GATA3fl/fl). p<0.05. Data represent mean±SEM.

FIG. 6. Association of the TLR4 Asp299Gly heterozygous genotype in RSV-infected infants with mild (white bars) and severe (black bars) bronchiolitis in groups exposed to high and low environmental levels of LPS in 2003-2006.

FIGS. 7A-B. (FIG. 7A) CD14 C-159T and (FIG. 7B) CD14 C-550T alleles in RSV-infected infants with severe (black bars) and mild (white bars) bronchiolitis in groups exposed to low and high environmental levels of LPS. P=NS for both alleles.

FIGS. 8A-F. Total IgE levels in serum of RSV-infected infants (FIG. 8A) with severe (black bar) and mild (white bar) bronchiolitis. P=NS, (FIG. 8B) exposed to low (black bar) and high (white bar) environmental levels of LPS. P=0.06, (FIG. 8C) with two major alleles (black bar) and infants with one TLR4D299G allele (white bar). P=NS. (FIG. 8D) IL-4/IFN-γ ratio in PBMC from RSV-infected infants with severe (black bar) and mild (white bar) bronchiolitis. P=NS. (FIGS. 8E-F) association of the IL4 C-590T and IL13 A-445G SNP in RSV-infected infants with mild (white bar) and severe (black bar) bronchiolitis in both environments. P=NS.

### DESCRIPTION OF ILLUSTRATIVE EMBODIMENTS

While 30-70% of RSV-infected infants develop bronchiolitis, only 1-3% require hospitalization. The mechanism for the differential severity of illness among healthy term infants is not well understood. Virus titer, inflammation, and Th2 bias in the lungs are proposed explanations. Toll-like receptor 4 (TLR4) is associated with each of these disease phenotypes, but its role in pathogenesis is controversial.

The inventors now have shown in two independent populations of 418 and 433 infants with RSV bronchiolitis that severity is determined by an interaction of TLR4 genotype with environments rich in lipopolysaccharide (LPS), and effected through GATA3/T-bet ratios expressed as high interleukin-4(IL-4)/interferon-γ(IFNγ) ratios in respiratory secretions. The mechanistic role of these molecules is confirmed in a mouse model of RSV that replicates the infant phenotype.

This investigation integrates environmental, epidemiological, viral, immune, genetic, and clinical findings and provides a framework for additional translational research on RSV bronchiolitis. Furthermore, this study identifies a group of full term infants without obvious phenotypic characteristics that may be highly susceptible to RSV: TLR4^{+/-} infants from environments with low levels of LPS may have hospitalization rates similar to those of very low birth premature babies. But while premature babies <1,500 g at birth are ˜1.5% of the population in western, industrialized societies, TLR4^{+/-} term infants represent ˜10%.

### I. DEFINITIONS

As used herein, an “allele” is one of a pair or series of genetic variants of a polymorphism at a specific genomic location. A “response allele” is an allele that is associated with altered response to a treatment. Where a SNP is biallelic, both alleles will be response alleles (e.g., one will be associated with a positive response, while the other allele is associated with no or a negative response, or some variation thereof).

As used herein, “genotype” refers to the diploid combination of alleles for a given genetic polymorphism. A homozygous subject carries two copies of the same allele and a heterozygous subject carries two different alleles.

As used herein, a “haplotype” is one or a set of signature genetic changes (polymorphisms) that are normally grouped closely together on the DNA strand, and are inherited as a group; the polymorphisms are also referred to herein as “markers.” A “haplotype” as used herein is information regarding the presence or absence of one or more genetic markers in a given chromosomal region in a subject. A haplotype can consist of a variety of genetic markers, including indels (insertions or deletions of the DNA at particular locations on the chromosome); single nucleotide polymorphisms (SNPs) in which a particular nucleotide is changed; microsatellites; and minisatellites.

Microsatellites (sometimes referred to as a variable number of tandem repeats or VNTRs) are short segments of DNA that have a repeated sequence, usually about 2 to 5 nucleotides long (e.g., a CA nucleotide pair repeated three times), that tend to occur in non-coding DNA. Changes in the microsatellites sometimes occur during the genetic recombination of sexual reproduction, increasing or decreasing the number of repeats found at an allele, changing the length of the allele. Microsatellite markers are stable, polymorphic, easily analyzed and occur regularly throughout the genome, making them especially suitable for genetic analysis.

“Copy number variation” (CNV), as used herein, refers to variation from the normal diploid condition for a gene or polymorphism. Individual segments of human chromosomes can be deleted or duplicated such that the subject's two chromosomes carry fewer than two copies of the gene or polymorphism (a deletion or deficiency) or two or more copies (a duplication).

“Linkage disequilibrium” (LD) refers to when the observed frequencies of haplotypes in a population does not agree with haplotype frequencies predicted by multiplying together the frequency of individual genetic markers in each haplotype. When SNPs and other variations that comprise a given haplotype are in LD with one another, alleles at the different markers correlate with one another.

The term “chromosome” as used herein refers to a gene carrier of a cell that is derived from chromatin and comprises DNA and protein components (e.g., histones). The conventional internationally recognized individual human genome chromosome numbering identification system is employed herein. The size of an individual chromosome can vary from one type to another with a given multi-chromosomal genome and from one genome to another. In the case of the human genome, the entire DNA mass of a given chromosome is usually greater than about 100,000,000 base pairs. For example, the size of the entire human genome is about 3×10⁹ base pairs.

The term “gene” refers to a DNA sequence in a chromosome that encodes a product (either RNA or its translation product, a polypeptide). A gene contains a coding region and includes regions preceding and following the coding region (termed respectively “leader” and “trailer”). The coding region is comprised of a plurality of coding segments (“exons”) and intervening sequences (“introns”) between individual coding segments.

The term “probe” refers to an oligonucleotide. A probe can be single stranded at the time of hybridization to a target. As used herein, probes include primers, i.e., oligonucleotides that can be used to prime a reaction, e.g., a PCR reaction.

The term “label” or “label containing moiety” refers in a moiety capable of detection, such as a radioactive isotope or group containing the same, and nonisotopic labels, such as enzymes, biotin, avidin, streptavidin, digoxygenin, luminescent agents, dyes, haptens, and the like. Luminescent agents, depending upon the source of exciting energy, can be classified as radioluminescent, chemiluminescent, bioluminescent, and photoluminescent (including fluorescent and phosphorescent). A probe described herein can be bound, e.g., chemically bound to label-containing moieties or can be suitable to be so bound. The probe can be directly or indirectly labeled.

The term “direct label probe” (or “directly labeled probe”) refers to a nucleic acid probe whose label after hybrid formation with a target is detectable without further reactive processing of the hybrid. The term “indirect label probe” (or “indirectly labeled probe”) refers to a nucleic acid probe whose label after hybrid formation with a target is further reacted in subsequent processing with one or more reagents to associate therewith one or more moieties that finally result in a detectable entity.

The terms “target,” “DNA target,” or “DNA target region” refers to a nucleotide sequence that occurs at a specific chromosomal location. Each such sequence or portion is preferably, at least partially, single stranded (e.g., denatured) at the time of hybridization. When the target nucleotide sequences are located only in a single region or fraction of a given chromosome, the term “target region” is sometimes used. Targets for hybridization can be derived from specimens that include, but are not limited to, chromosomes or regions of chromosomes in normal, diseased or malignant human cells, either interphase or at any state of meiosis or mitosis, and either extracted or derived from living or postmortem tissues, organs or fluids; germinal cells including sperm and egg cells, or cells from zygotes, fetuses, or embryos, or chorionic or amniotic cells, or cells from any other germinating body; cells grown in vitro, from either long-term or short-term culture, and either normal, immortalized or transformed; inter- or intraspecific hybrids of different types of cells or differentiation states of these cells; individual chromosomes or portions of chromosomes, or translocated, deleted or other damaged chromosomes, isolated by any of a number of means known to those with skill in the art, including libraries of such chromosomes cloned and propagated in prokaryotic or other cloning vectors, or amplified in vitro by means well known to those with skill; or any forensic material, including but not limited to blood, or other samples.

The term “hybrid” refers to the product of a hybridization procedure between a probe and a target.

The term “hybridizing conditions” has general reference to the combinations of conditions that are employable in a given hybridization procedure to produce hybrids, such conditions typically involving controlled temperature, liquid phase, and contact between a probe (or probe composition) and a target. Conveniently and preferably, at least one denaturation step precedes a step wherein a probe or probe composition is contacted with a target. Guidance for performing hybridization reactions can be found in Ausubel et al. (2003), Current Protocols in Molecular Biology, John Wiley & Sons, N.Y. (2003), 6.3.1-6.3.6. Aqueous and nonaqueous methods are described in that reference and either can be used. Hybridization conditions referred to herein are a 50% formamide, 2×SSC wash for 10 minutes at 45° C. followed by a 2×SSC wash for 10 minutes at 37° C.

The term “SNP” stands for single nucleotide polymorphism, and in particular refers to rs4986790 (C_{—}11722238; Asp299Gly, rs4986790) and rs4986791 (C_{—}11722237; Ile399Thr).

Calculations of “identity” between two sequences can be performed as follows. The sequences are aligned for optimal comparison purposes (e.g., gaps can be introduced in one or both of a first and a second nucleic acid sequence for optimal alignment and non-identical sequences can be disregarded for comparison purposes). The length of a sequence aligned for comparison purposes is at least 30% (e.g., at least 40%, 50%, 60%, 70%, 80%, 90% or 100%) of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In some embodiments, the percent identity between two nucleotide sequences is determined using the GAP program in the GCG software package, using a Blossum 62 scoring matrix with a gap penalty of 12, a gap extend penalty of 4, and a frameshift gap penalty of 5.

As used herein, the term “substantially identical” is used to refer to a first nucleotide sequence that contains a sufficient number of identical nucleotides to a second nucleotide sequence such that the first and second nucleotide sequences have similar activities. Nucleotide sequences that are substantially identical are at least 80% (e.g., 85%, 90%, 95%, 97% or more) identical.

The term “nonspecific binding DNA” refers to DNA that is complementary to DNA segments of a probe, which DNA occurs in at least one other position in a genome, outside of a selected chromosomal target region within that genome. An example of nonspecific binding DNA comprises a class of DNA repeated segments whose members commonly occur in more than one chromosome or chromosome region. Such common repetitive segments tend to hybridize to a greater extent than other DNA segments that are present in probe composition.

As used herein, the term “stratification” refers to the creation of a distinction between subjects on the basis of a characteristic or characteristics of the subjects. Generally, in the context of clinical trials, the distinction is used to distinguish responses or effects in different sets of patients distinguished according to the stratification parameters. In some embodiments, stratification includes distinction of subject groups based on the presence or absence of particular markers or alleles described herein. The stratification can be performed, e.g., in the course of analysis, or can be used in creation of distinct groups or in other ways.

### II. METHODS OF PREDICTING RSV INFECTION SEVERITY

Described herein are a variety of methods for predicting the severity of a subject's RSV infection based in part on the presence or absence of an allele defined by the SNP designated rs4986790 and rs4986791. As used herein, “determining the identity of an allele” includes obtaining information regarding the identity (i.e., of a specific nucleotide), presence or absence of one or more specific alleles in a subject. Determining the identity of an allele can, but need not, include obtaining a sample comprising DNA from a subject, and/or assessing the identity, presence or absence of one or more genetic markers in the sample. The individual or organization who determines the identity of the allele need not actually carry out the physical analysis of a sample from a subject; the methods can include using information obtained by analysis of the sample by a third party. Thus the methods can include steps that occur at more than one site. For example, a sample can be obtained from a subject at a first site, such as at a healthcare provider, or at the subject's home in the case of a self-testing kit. The sample can be analyzed at the same or a second site, e.g., at a laboratory or other testing facility.

Determining the identity of an allele can also include or consist of reviewing a subject's medical history, where the medical history includes information regarding the identity, presence or absence of one or more response alleles in the subject, e.g., results of a genetic test.

In some embodiments, to determine the identity of an allele described herein, a biological sample that includes nucleated cells (such as blood, a cheek swab or mouthwash) is prepared and analyzed for the presence or absence of preselected markers. Such diagnoses may be performed by diagnostic laboratories, or, alternatively, diagnostic kits can be manufactured and sold to health care providers or to private individuals for self-diagnosis. Diagnostic or prognostic tests can be performed as described herein or using well known techniques, such as described in U.S. Pat. No. 5,800,998.

Results of these tests, and optionally interpretive information, can be returned to the subject, the health care provider or to a third party payor. The results can be used in a number of ways. The information can be, e.g., communicated to the tested subject, e.g., with a prognosis and optionally interpretive materials that help the subject understand the test results and prognosis. The information can be used, e.g., by a health care provider, to determine whether to administer a specific drug, or whether a subject should be assigned to a specific category, e.g., a category associated with a specific disease endophenotype, or with drug response or non-response. The information can be used, e.g., by a third party payor such as a healthcare payer (e.g., insurance company or HMO) or other agency, to determine whether or not to reimburse a health care provider for services to the subject, or whether to approve the provision of services to the subject. For example, the healthcare payer may decide to reimburse a health care provider for treatments for an SSD if the subject has a particular response allele. As another example, a drug or treatment may be indicated for individuals with a certain allele, and the insurance company would only reimburse the health care provider (or the insured individual) for prescription or purchase of the drug if the insured individual has that response allele. The presence or absence of the response allele in a patient may be ascertained by using any of the methods described herein.

A. TLR4 and Risk Alleles

Toll-like receptor 4 is a protein that in humans is encoded by the TLR4 gene. TLR 4 is a toll-like receptor. It detects lipopolysaccharide from Gram-negative bacteria and is thus important in the activation of the innate immune system. TLR 4 has also been designated as CD284 (cluster of differentiation 284). The protein encoded by this gene is a member of the Toll-like receptor (TLR) family, which plays a fundamental role in pathogen recognition and activation of innate immunity. TLRs are highly conserved from Drosophila to humans and share structural and functional similarities. They recognize pathogen-associated molecular patterns (PAMPs) that are expressed on infectious agents, and mediate the production of cytokines necessary for the development of effective immunity.

The various TLRs exhibit different patterns of expression. This receptor is most abundantly expressed in placenta, and in myelomonocytic subpopulation of the leukocytes. It cooperates with LY96 and CD14 to mediate in signal transduction events induced by lipopolysaccharide (LPS) found in most gram-negative bacteria. Mutations in this gene have been associated with differences in LPS responsiveness. Several transcript variants of this gene have been found, but the protein-coding potential of most of them is uncertain. TLR 4 has been shown to interact with Lymphocyte antigen 96, Myd88 and TOLLIP.

Intracellular trafficking of TLR4 is dependent on the GTPase Rab-11a, and knock-down of Rab-11a results in hampered TLR4 recruitment to E. coli-containing phagosomes and subsequent reduced signal transduction through the MyD88-independent pathway.

rs4986790 (+896A>G) resides in the coding region for TLR4 and results in an Asp to Gly change at residue 299. It is often studied along with a co-segregating SNP known as Thr399Ile, rs4986791. TLR4 encodes a receptor involved in the innate immune response. Together, these SNPs have been reported to be associated with a wide variety of both infectious and non-infectious diseases, although there are conflicting or even contradictory results also reported in some cases. The risk allele for rs4986790 is (G). The Gly allele of TLR4 rs4986790 gene polymorphism has been implicated in reduction of risk of atherosclerosis, premature birth, vaginal infection, septic shock, severity of asthma attack, and Alzheimer's disease. Tal et al. (2004) examined the association of the TLR4 mutations (Asp299Gly and Thr399Ile) and the CD14/-159 polymorphism with severe RSV bronchiolitis. They found that each of the TLR4 mutations, either alone or in co-segregation, were associated with severe RSV bronchiolitis. No association between the CD14/-159 polymorphism and RSV bronchiolitis was found. These findings, however, are not entirely consistent with the results from similar studies (see Supp. Table 1).

B. Markers in Linkage Disequilibrium (LD)

Linkage disequilibrium (LD) is a measure of the degree of association between alleles in a population. One of skill in the art will appreciate that alleles involving markers in LD with the polymorphisms described herein can also be used in a similar manner to those described herein. In particular, rs4986791 (Thr399Ile) is known to co-segregate

Methods of calculating LD are known in the art (see, e.g., Morton et al., 2001; Tapper et al., 2005; Maniatis et al., 2002). Thus, in some cases, the methods can include analysis of polymorphisms that are in LD with a polymorphism described herein. Methods are known in the art for identifying such polymorphisms; for example, the International HapMap Project provides a public database that can be used, see hapmap.org, as well as The International HapMap Consortium (2003) and The International HapMap Consortium (2005). Generally, it will be desirable to use a HapMap constructed using data from individuals who share ethnicity with the subject. For example, a HapMap for Caucasians would ideally be used to identify markers in LD with an exemplary marker described herein for use in genotyping a subject of Caucasian descent.

Alternatively, methods described herein can include analysis of polymorphisms that show a correlation coefficient (r²) of value >0.5 with the markers described herein. Results can be obtained from on line public resources such as HapMap.org on the World Wide Web. The correlation coefficient is a measure of LD, and reflects the degree to which alleles at two loci (for example, two SNPs) occur together, such that an allele at one SNP position can predict the correlated allele at a second SNP position, in the case where r² is >0.5.

C. Methods of Determining Genotypes

The methods described herein include determining the identity, e.g., the specific nucleotide, presence or absence, of a SNP associated with a severe RSV infection. Samples that are suitable for use in the methods described herein contain genetic material, e.g., genomic DNA (gDNA). Genomic DNA is typically extracted from biological samples such as blood or mucosal scrapings of the lining of the mouth, but can be extracted from other biological samples including urine or expectorant. The sample itself will typically include nucleated cells (e.g., blood or buccal cells) or tissue removed from the subject. The subject can be an adult, child, fetus, or embryo. In some embodiments, the sample is obtained prenatally, either from a fetus or embryo or from the mother (e.g., from fetal or embryonic cells in the maternal circulation). Methods and reagents are known in the art for obtaining, processing, and analyzing samples. In some embodiments, the sample is obtained with the assistance of a health care provider, e.g., to draw blood. In some embodiments, the sample is obtained without the assistance of a health care provider, e.g., where the sample is obtained non-invasively, such as a sample comprising buccal cells that is obtained using a buccal swab or brush, or a mouthwash sample.

In some cases, a biological sample may be processed for DNA isolation. For example, DNA in a cell or tissue sample can be separated from other components of the sample. Cells can be harvested from a biological sample using standard techniques known in the art. For example, cells can be harvested by centrifuging a cell sample and resuspending the pelleted cells. The cells can be resuspended in a buffered solution such as phosphate-buffered saline (PBS). After centrifuging the cell suspension to obtain a cell pellet, the cells can be lysed to extract DNA, e.g., gDNA. See, e.g., Ausubel et al. (2003). The sample can be concentrated and/or purified to isolate DNA. All samples obtained from a subject, including those subjected to any sort of further processing, are considered to be obtained from the subject. Routine methods can be used to extract genomic DNA from a biological sample, including, for example, phenol extraction. Alternatively, genomic DNA can be extracted with kits such as the QIAamp® Tissue Kit (Qiagen, Chatsworth, Calif.) and the Wizard® Genomic DNA purification kit (Promega). Non-limiting examples of sources of samples include urine, blood, and tissue.

The presence or absence of the SNP can be determined using methods known in the art. For example, gel electrophoresis, capillary electrophoresis, size exclusion chromatography, sequencing, and/or arrays can be used to detect the presence or absence of specific response alleles. Amplification of nucleic acids, where desirable, can be accomplished using methods known in the art, e.g., PCR. In one example, a sample (e.g., a sample comprising genomic DNA), is obtained from a subject. The DNA in the sample is then examined to determine the identity of an allele as described herein, i.e., by determining the identity of one or more alleles associated with a selected response. The identity of an allele can be determined by any method described herein, e.g., by sequencing or by hybridization of the gene in the genomic DNA, RNA, or cDNA to a nucleic acid probe, e.g., a DNA probe (which includes cDNA and oligonucleotide probes) or an RNA probe. The nucleic acid probe can be designed to specifically or preferentially hybridize with a particular polymorphic variant.

Other methods of nucleic acid analysis can include direct manual sequencing (Church and Gilbert, 1988; Sanger et al., 1977; U.S. Pat. No. 5,288,644); automated fluorescent sequencing; single-stranded conformation polymorphism assays (SSCP) (Schafer et al., 1995); clamped denaturing gel electrophoresis (CDGE); two-dimensional gel electrophoresis (2DGE or TDGE); conformational sensitive gel electrophoresis (CSGE); denaturing gradient gel electrophoresis (DGGE) (Sheffield et al., 1989); denaturing high performance liquid chromatography (DHPLC) (Underhill et al., 1997); infrared matrix-assisted laser desorption/ionization (IR-MALDI) mass spectrometry (WO 99/57318); mobility shift analysis (Orita et al., 1989); restriction enzyme analysis (Flavell et al., 1978; Geever et al., 1981); quantitative real-time PCR (Raca et al., 2004); heteroduplex analysis; chemical mismatch cleavage (CMC) (Cotton et al., 1985); RNase protection assays (Myers et al., 1985); use of polypeptides that recognize nucleotide mismatches, e.g., E. coli mutS protein; allele-specific PCR, and combinations of such methods. See, e.g., U.S. Patent Publication No. 2004/0014095, which is incorporated herein by reference in its entirety.

Sequence analysis can also be used to detect specific polymorphic variants. For example, polymorphic variants can be detected by sequencing exons, introns, 5′ untranslated sequences, or 3′ untranslated sequences. A sample comprising DNA or RNA is obtained from the subject. PCR or other appropriate methods can be used to amplify a portion encompassing the polymorphic site, if desired. The sequence is then ascertained, using any standard method, and the presence of a polymorphic variant is determined. Real-time pyrophosphate DNA sequencing is yet another approach to detection of polymorphisms and polymorphic variants (Alderborn et al., 2000). Additional methods include, for example, PCR amplification in combination with denaturing high performance liquid chromatography (dHPLC) (Underhill et al., 1997).

PCR® refers to procedures in which target nucleic acid (e.g., genomic DNA) is amplified in a manner similar to that described in U.S. Pat. No. 4,683,195, and subsequent modifications of the procedure described therein. Generally, sequence information from the ends of the region of interest or beyond are used to design oligonucleotide primers that are identical or similar in sequence to opposite strands of a potential template to be amplified. See e.g., PCR Primer: A Laboratory Manual, Dieffenbach and Dveksler, (Eds.); McPherson et al., 2000; Mattila et al., 1991; Eckert et al., 1991; PCR (eds. McPherson et al., IRL Press, Oxford); and U.S. Pat. No. 4,683,202. Other amplification methods that may be employed include the ligase chain reaction (LCR) (Wu and Wallace, 1989; Landegren et al., 1988), transcription amplification (Kwoh et al., 1989), self-sustained sequence replication (Guatelli et al., 1990), and nucleic acid based sequence amplification (NASBA). Guidelines for selecting primers for PCR amplification are well known in the art. See, e.g., McPherson et al. (2000). A variety of computer programs for designing primers are available, e.g., ‘Oligo’ (National Biosciences, Inc, Plymouth Minn.), MacVector (Kodak/IBI), and the GCG suite of sequence analysis programs (Genetics Computer Group, Madison, Wis. 53711).

In some cases, PCR conditions and primers can be developed that amplify a product only when the variant allele is present or only when the wild-type allele is present (MSPCR or allele-specific PCR). For example, patient DNA and a control can be amplified separately using either a wild-type primer or a primer specific for the variant allele. Each set of reactions is then examined for the presence of amplification products using standard methods to visualize the DNA. For example, the reactions can be electrophoresed through an agarose gel and the DNA visualized by staining with ethidium bromide or other DNA intercalating dye. In DNA samples from heterozygous patients, reaction products would be detected in each reaction.

In some embodiments, a peptide nucleic acid (PNA) probe can be used instead of a nucleic acid probe in the hybridization methods described above. PNA is a DNA mimetic with a peptide-like, inorganic backbone, e.g., N-(2-aminoethyl)glycine units, with an organic base (A, G, C, T or U) attached to the glycine nitrogen via a methylene carbonyl linker (see, e.g., Nielsen et al., 1994). The PNA probe can be designed to specifically hybridize to a nucleic acid comprising a polymorphic variant.

In some cases, allele-specific oligonucleotides can also be used to detect the presence of a polymorphic variant. For example, polymorphic variants can be detected by performing allele-specific hybridization or allele-specific restriction digests. Allele specific hybridization is an example of a method that can be used to detect sequence variants, including complete genotypes of a subject (e.g., a mammal such as a human). See Stoneking et al., 1991; Prince et al., 2001. An “allele-specific oligonucleotide” (also referred to herein as an “allele-specific oligonucleotide probe”) is an oligonucleotide that is specific for particular a polymorphism can be prepared using standard methods (see, Ausubel et al., 2003). Allele-specific oligonucleotide probes typically can be approximately 10-50 base pairs, preferably approximately 15-30 base pairs, that specifically hybridizes to a nucleic acid region that contains a polymorphism. Hybridization conditions are selected such that a nucleic acid probe can specifically bind to the sequence of interest, e.g., the variant nucleic acid sequence. Such hybridizations typically are performed under high stringency as some sequence variants include only a single nucleotide difference. In some cases, dot-blot hybridization of amplified oligonucleotides with allele-specific oligonucleotide (ASO) probes can be performed. See, for example, Saiki et al., 1986.

In some embodiments, allele-specific restriction digest analysis can be used to detect the existence of a polymorphic variant of a polymorphism, if alternate polymorphic variants of the polymorphism result in the creation or elimination of a restriction site. Allele-specific restriction digests can be performed in the following manner. A sample containing genomic DNA is obtained from the individual and genomic DNA is isolated for analysis. For nucleotide sequence variants that introduce a restriction site, restriction digest with the particular restriction enzyme can differentiate the alleles. In some cases, polymerase chain reaction (PCR) can be used to amplify a region comprising the polymorphic site, and restriction fragment length polymorphism analysis is conducted (see, Ausubel et al., 2003). The digestion pattern of the relevant DNA fragment indicates the presence or absence of a particular polymorphic variant of the polymorphism and is therefore indicative of the subject's response allele. For sequence variants that do not alter a common restriction site, mutagenic primers can be designed that introduce a restriction site when the variant allele is present or when the wild-type allele is present. For example, a portion of a nucleic acid can be amplified using the mutagenic primer and a wild-type primer, followed by digest with the appropriate restriction endonuclease.

In some embodiments, fluorescence polarization template-directed dye-terminator incorporation (FP-TDI) is used to determine which of multiple polymorphic variants of a polymorphism is present in a subject (Chen et al., 1999). Rather than involving use of allele-specific probes or primers, this method employs primers that terminate adjacent to a polymorphic site, so that extension of the primer by a single nucleotide results in incorporation of a nucleotide complementary to the polymorphic variant at the polymorphic site.

In some cases, DNA containing an amplified portion may be dot-blotted, using standard methods (see Ausubel et al., 2003), and the blot contacted with the oligonucleotide probe. The presence of specific hybridization of the probe to the DNA is then detected. Specific hybridization of an allele-specific oligonucleotide probe (specific for a polymorphic variant indicative of a predicted response to a method of treating an SSD) to DNA from the subject is indicative of a subject's response allele.

The methods can include determining the genotype of a subject with respect to both copies of the polymorphic site present in the genome (i.e., both alleles). For example, the complete genotype may be characterized as −/−, as −/+, or as +/+, where a minus sign indicates the presence of the reference or wild-type sequence at the polymorphic site, and the plus sign indicates the presence of a polymorphic variant other than the reference sequence. If multiple polymorphic variants exist at a site, this can be appropriately indicated by specifying which ones are present in the subject. Any of the detection means described herein can be used to determine the genotype of a subject with respect to one or both copies of the polymorphism present in the subject's genome.

Methods of nucleic acid analysis to detect polymorphisms and/or polymorphic variants can include, e.g., microarray analysis. Hybridization methods, such as Southern analysis, Northern analysis, or in situ hybridizations, can also be used (see, Ausubel et al., 2003). To detect microdeletions, fluorescence in situ hybridization (FISH) using DNA probes that are directed to a putatively deleted region in a chromosome can be used. For example, probes that detect all or a part of a microsatellite marker can be used to detect microdeletions in the region that contains that marker.

In some embodiments, it is desirable to employ methods that can detect the presence of multiple polymorphisms (e.g., polymorphic variants at a plurality of polymorphic sites) in parallel or substantially simultaneously. Oligonucleotide arrays represent one suitable means for doing so. Other methods, including methods in which reactions (e.g., amplification, hybridization) are performed in individual vessels, e.g., within individual wells of a multi-well plate or other vessel may also be performed so as to detect the presence of multiple polymorphic variants (e.g., polymorphic variants at a plurality of polymorphic sites) in parallel or substantially simultaneously according to the methods provided herein.

Nucleic acid probes can be used to detect and/or quantify the presence of a particular target nucleic acid sequence within a sample of nucleic acid sequences, e.g., as hybridization probes, or to amplify a particular target sequence within a sample, e.g., as a primer. Probes have a complimentary nucleic acid sequence that selectively hybridizes to the target nucleic acid sequence. In order for a probe to hybridize to a target sequence, the hybridization probe must have sufficient identity with the target sequence, i.e., at least 70% (e.g., 80%, 90%, 95%, 98% or more) identity to the target sequence. The probe sequence must also be sufficiently long so that the probe exhibits selectivity for the target sequence over non-target sequences. For example, the probe will be at least 20 (e.g., 25, 30, 35, 50, 100, 200, 300, 400, 500, 600, 700, 800, 900 or more) nucleotides in length. In some embodiments, the probes are not more than 30, 50, 100, 200, 300, 500, 750, or 1000 nucleotides in length. Probes are typically about 20 to about 1×10⁶ nucleotides in length. Probes include primers, which generally refers to a single-stranded oligonucleotide probe that can act as a point of initiation of template-directed DNA synthesis using methods such as PCR (polymerase chain reaction), LCR (ligase chain reaction), etc., for amplification of a target sequence.

The probe can be a test probe such as a probe that can be used to detect polymorphisms in a region described herein (e.g., an allele associated with treatment response as described herein). In some embodiments, the probe can bind to another marker sequence associated with SZ, SPD, or SD as described herein or known in the art.

Control probes can also be used. For example, a probe that binds a less variable sequence, e.g., repetitive DNA associated with a centromere of a chromosome, can be used as a control. Probes that hybridize with various centromeric DNA and locus-specific DNA are available commercially, for example, from Vysis, Inc. (Downers Grove, Ill.), Molecular Probes, Inc. (Eugene, Oreg.), or from Cytocell (Oxfordshire, UK). Probe sets are available commercially such from Applied Biosystems, e.g., the Assays-on-Demand SNP kits Alternatively, probes can be synthesized, e.g., chemically or in vitro, or made from chromosomal or genomic DNA through standard techniques. For example, sources of DNA that can be used include genomic DNA, cloned DNA sequences, somatic cell hybrids that contain one, or a part of one, human chromosome along with the normal chromosome complement of the host, and chromosomes purified by flow cytometry or microdissection. The region of interest can be isolated through cloning, or by site-specific amplification via the polymerase chain reaction (PCR). See, for example, Nath and Johnson, (1998); Wheeless et al., (1994); U.S. Pat. No. 5,491,224.

In some embodiments, the probes are labeled, e.g., by direct labeling, with a fluorophore, an organic molecule that fluoresces after absorbing light of lower wavelength/higher energy. A directly labeled fluorophore allows the probe to be visualized without a secondary detection molecule. After covalently attaching a fluorophore to a nucleotide, the nucleotide can be directly incorporated into the probe with standard techniques such as nick translation, random priming, and PCR labeling. Alternatively, deoxycytidine nucleotides within the probe can be transaminated with a linker. The fluorophore then is covalently attached to the transaminated deoxycytidine nucleotides. See, e.g., U.S. Pat. No. 5,491,224.

Fluorophores of different colors can be chosen such that each probe in a set can be distinctly visualized. For example, a combination of the following fluorophores can be used: 7-amino-4-methylcoumarin-3-acetic acid (AMCA), TEXAS RED™ (Molecular Probes, Inc., Eugene, Oreg.), 5-(and -6)-carboxy-X-rhodamine, lissamine rhodamine B, 5-(and -6)-carboxyfluorescein, fluorescein-5-isothiocyanate (FITC), 7-diethylaminocoumarin-3-carboxylic acid, tetramethylrhodamine-5-(and -6)-isothiocyanate, 5-(and -6)-carboxytetramethylrhodamine, 7-hydroxycoumarin-3-carboxylic acid, 6-[fluorescein 5-(and -6)-carboxamido]hexanoic acid, N-(4,4-difluoro-5,7-dimethyl-4-bora-3a,4a diaza-3-indacenepropionic acid, eosin-5-isothiocyanate, erythrosin-5-isothiocyanate, and CASCADE™ blue acetylazide (Molecular Probes, Inc., Eugene, Oreg.). Fluorescently labeled probes can be viewed with a fluorescence microscope and an appropriate filter for each fluorophore, or by using dual or triple band-pass filter sets to observe multiple fluorophores. See, for example, U.S. Pat. No. 5,776,688. Alternatively, techniques such as flow cytometry can be used to examine the hybridization pattern of the probes. Fluorescence-based arrays are also known in the art.

In other embodiments, the probes can be indirectly labeled with, e.g., biotin or digoxygenin, or labeled with radioactive isotopes such as ³²P and ³H. For example, a probe indirectly labeled with biotin can be detected by avidin conjugated to a detectable marker. For example, avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase.

### III. RESPIRATORY SYNCYTIAL VIRUS

Human respiratory syncytial virus (RSV) is a virus that causes respiratory tract infections. It is a major cause of lower respiratory tract infections and hospital visits during infancy and childhood. A prophylactic medication (not a vaccine) exists for preterm (under 35 weeks gestation) infants, infants with certain congenital heart defects (CHD) or bronchopulmonary dysplasia (BPD), and infants with congenital malformations of the airway. Treatment is limited to supportive care (for example C-PAP), including oxygen therapy.

In temperate climates there is an annual epidemic during the winter months. In tropical climates, infection is most common during the rainy season. In the United States, 60% of infants are infected during their first RSV season, and nearly all children will have been infected with the virus by 2-3 years of age. Of those infected with RSV, 2-3% will develop bronchiolitis, necessitating hospitalization. Natural infection with RSV induces protective immunity which wanes over time—possibly more so than other respiratory viral infections—and thus people can be infected multiple times. Sometimes an infant can become symptomatically infected more than once, even within a single RSV season. Severe RSV infections have increasingly been found among elderly patients. Young adults can be re-infected every five to seven years, with symptoms looking like a sinus infection or a cold (infections can also be asymptomatic).

RSV is a negative-sense, single-stranded RNA virus of the family Paramyxoviridae, which includes common respiratory viruses such as those causing measles and mumps. RSV is a member of the paramyxovirus subfamily Pneumovirinae. Its name comes from the fact that F proteins on the surface of the virus cause the cell membranes on nearby cells to merge, forming syncytia.

The incubation time is 4-5 days. For adults, RSV produces only mild symptoms, often indistinguishable from common colds and minor illnesses. The Centers for Disease Control consider RSV to be the “most common cause of bronchiolitis (inflammation of the small airways in the lung) and pneumonia in children under 1 year of age in the United States”. For some children, RSV can cause bronchiolitis, leading to severe respiratory illness requiring hospitalization and, rarely, causing death. This is more likely to occur in patients that are immunocompromised or infants born prematurely. Other RSV symptoms common among infants include listlessness, poor or diminished appetite, and a possible fever.

Recurrent wheezing and asthma are more common among individuals who suffered severe RSV infection during the first few months of life than among those who did controls; whether RSV infection sets up a process that leads to recurrent wheezing or whether those already predisposed to asthma are more likely to become severely ill with RSV has yet to be determined. In fact, which infants will go on to develop frequent wheezing and asthma after suffering an RSV infection early in life is unclear. However, these data suggest that individuals heterozygous for the 299 &/or 399 alleles are more likely to develop recurrent wheezing when infected with RSV. Symptoms of pneumonia in immunocompromised patients such as in transplant patients and especially bone marrow transplant patients should be evaluated to rule out RSV infection. This can be done by means of PCR testing for RSV nucleic acids in peripheral blood samples if all other infectious processes have been ruled out or if it is highly suspicious for RSV such as a recent exposure to a known source of RSV infection.

The genome is ˜15,000 nucleotides in length and is composed of a single strand of RNA with negative polarity. It has 10 genes encoding 11 proteins—there are 2 open reading frames of M2. The genome is transcribed sequentially from NS1 to L with reduction in expression levels along its length. NS1 and NS2 inhibit type I interferon activity. N encodes nucleocapsid protein that associates with the genomic RNA forming the nucleocapsid. M encodes the Matrix protein required for viral assembly. SH, G and F form the viral coat. The G protein is a surface protein that is heavily glycosylated. It functions as the attachment protein. The F protein is another important surface protein; F mediates fusion, allowing entry of the virus into the cell cytoplasm and also allowing the formation of syncytia. The F protein is homologous in both subtypes of RSV; antibodies directed at the F protein are neutralizing. In contrast, the G protein differs considerably between the two subtypes. M2 is the second matrix protein also required for transcription and encodes M2-1 (elongation factor) and M2-2 (transcription regulation). M2 contains CD8 epitopes. L encodes the RNA polymerase. The phosphoprotein P is a cofactor for the L protein. The atomic structure is now available for two of these proteins: N and M.

RSV spreads easily by direct contact, and can remain viable for a half an hour or more on hands or for up to 5 hours on countertops. As the virus is ubiquitous in all parts of the world, avoidance of infection is not possible. Epidemiologically, a vaccine would be the best answer, but at present no vaccine exists. Some of the most promising candidates are based on temperature sensitive mutants which have targeted genetic mutations to reduce virulence.

However, palivizumab (brand name Synagis manufactured by Medlmmune), a moderately effective prophylactic drug is available for infants at high risk. Palivizumab is a monoclonal antibody directed against RSV surface fusion protein. It is given by monthly injections, which are begun just prior to the RSV season and are usually continued for five months. RSV prophylaxis is indicated for infants that are premature or have either cardiac or lung disease, but the cost of prevention limits use in many parts of the world. An antiviral drug—Ribavirin—is licensed for use, but its efficacy is limited. Scientists are attempting to develop a recombinant human respiratory syncytial virus vaccine suitable for intranasal instillation. Tests for determining the safety and level of resistance that can be achieved by the vaccine are being conducted in animals and humans.

Studies of nebulized hypertonic saline have shown that the use of nebulized 3% HS is a safe, inexpensive, and effective treatment for infants hospitalized with moderately severe viral bronchiolitis where respiratory syncytial virus (RSV) accounts for the majority of viral bronchiolitis cases. One study noted a 26% reduction in length of stay: 2.6±1.9 days, compared with 3.5±2.9 days in the normal-saline treated group (p=0.05). Supportive care includes fluids and oxygen until the illness runs its course. Salbutamol may be used in an attempt to relieve any bronchospasm if present. Increased airflow, humidified and delivered via nasal cannula, may be supplied in order to reduce the effort required for respiration. Adrenaline, bronchodilators, steroids, antibiotics, and ribavirin confer “no real benefit.”

RSV infection can be confirmed using Direct Fluorescent Antibody detection (DFA), Chromatographic rapid antigen detection or detection of viral RNA using RT-PCR. Quantification of viral load can be determined by Plaque Assay, antigen capture enzyme immunoassay (EIA), ELISA and HA, and quantification of antibody levels by HAI and Neutralization assay.

### IV. DETERMINATION OF LIPOPOLYSACCHARIDE EXPOSURE

In another aspect of the disclosure, it is necessary to assess a subject's exposure to lipopolysaccharide (LPS). This may be done directly, such as in detecting LPS, or it may be done indirectly or inferentially, such as by using a proxy or surrogate marker for exposure.

There are a number different ways to detect LPS in a sample from an environment, but the most often employed is the Limulus amebocyte lysate (LAL) assay that uses is an aqueous extract of blood cells (amoebocytes) from the horseshoe crab, Limulus polyphemus. LAL reacts with bacterial endotoxin or lipopolysaccharide (LPS), which is a membrane component of Gram negative bacteria. This reaction is the basis of the LAL test, which is used for the detection and quantification of bacterial endotoxins.

Fred Bang reported in 1956 that gram-negative bacteria, even if killed, will cause the blood of the horseshoe crab to turn into a semi-solid mass. It was later recognized that the animal's blood cells, mobile cells called amebocytes, contain granules with a clotting factor known as coagulogen; this is released outside the cell when bacterial endotoxin is encountered. The resulting coagulation is thought to contain bacterial infections in the animal's semi-closed circulatory system.

Blood is removed from the horseshoe crab's pericardium, the blood cells are separated from the serum using centrifugation, and are then placed in distilled water, which causes them to swell and burst (“lyse”). This releases the chemicals from the inside of the cell (the “lysate”), which is then purified and freeze-dried. To test a sample for endotoxins, it is mixed with lysate and water; endotoxins are present if coagulation occurs. There are three basic LAL test methodologies: gel-clot, turbidimetric, and chromogenic. The primary application for LAL is the testing of parenteral pharmaceuticals and medical devices that contact blood or cerebrospinal fluid. In the United States, the FDA has published a guideline for validation of the LAL test as an endotoxin test for such products.

The LAL cascade is also triggered by (1,3)-β-D-glucan. Both bacterial endotoxins and (1,3)-β-D-glucan are considered “Pathogen-Associated Molecular Patterns,” or PAMPS, substances which elicit inflammatory responses in mammals.

One of the most time consuming aspects of endotoxin testing using LAL is pretreating samples to overcome assay inhibition and enhancement. Agents such as EDTA and heparin are known to affect the assay if they are present in sufficient concentrations. All assays, independent of methodology are standardized using endotoxin in water. Therefore, unless the sample is water, some components of the solution may interfere with the LAL test such that the recovery of endotoxin is affected. If the product being tested causes the endotoxin recovery to be less than expected, the product is inhibitory to the LAL test. Products which cause higher than expected values are enhancing. Overcoming the inhibition and enhancement properties of a product is required by the FDA as part of the validation of the LAL test for use in the final release testing of injectables and medical devices. Proper endotoxin recovery must be proven before LAL can be used to release product.

In contrast, one may determine LPS exposure inferentially by examining various factors that are a reasonable proxy for LPS exposure. This will, in one form, involve obtaining personal information about the subject, typically by use of a patient questionnaire. The questionnaire will seek to identify factors that tend to indicate a less clean living environment, which in turn will indicate a higher level of LPS exposure. Obviously, factors directly leading to conclusion about cleanliness are highly relevant, but more general factors, such as socio-economic status, have been found sufficiently correlative to provide accurate prediction of LPS exposure. However, recent studies have shed doubts about the appropriateness of such inferences. For example, examination of inner city homes of families of low socioeconomic status found environments with low levels of LPS defying previous expectations. Therefore, direct methods for assessing LPS through LAL assays are preferable and state-of-the-art for these evaluations.

Endotoxin measurement is typically assessed by a very sensitive but not entirely specific assay using a natural extract (LAL) made from horseshoe crab blood, which is being supplanted by an assay made using a cloned protein from the horseshoe crab. The major difference between the assays is that the old natural extract contains a large number of proteins and its composition and sensitivity is variable from lot-to-lot; it can react with other molecules (e.g., β (1-3) D-glucans or cellulose); and can be inhibited or enhanced in its LPS reactivity by many compounds present in environmental samples. The recombinant factor C (rfC) assay should avoid many of these problems and has little lot-to-lot variability. In addition, LPS and other PAMP levels in the environment may be inferred indirectly through an evaluation of cytokine production or cell surface expression of activation markers upon exposure of immune cells (or cells engeneered to express only TLR4) to house dust.

### V. ARTICLES OF MANUFACTURE

Also provided herein are articles of manufacture comprising probes that hybridize to or prime near the region of human chromosome containing the SNP described herein. For example, any of the probes for detecting the SNP described herein can be combined with packaging material to generate articles of manufacture or kits. The kit can include one or more other elements including: instructions for use; and other reagents such as a label or an agent useful for attaching a label to the probe. Instructions for use can include instructions for treatment RSV in a method described herein. Other instructions can include instructions for attaching a label to the probe, instructions for performing analysis with the probe, and/or instructions for obtaining a sample to be analyzed from a subject. In some cases, the kit can include a labeled probe that hybridizes to a region of human chromosome as described herein.

The kit can also include one or more additional reference or control probes that hybridize to the same chromosome or another chromosome or portion thereof. A kit that includes additional probes can further include labels, e.g., one or more of the same or different labels for the probes. In other embodiments, the additional probe or probes provided with the kit can be a labeled probe or probes. When the kit further includes one or more additional probe or probes, the kit can further provide instructions for the use of the additional probe or probes. Kits for use in self-testing can also be provided. Such test kits can include devices and instructions that a subject can use to obtain a biological sample (e.g., buccal cells, blood) without the aid of a health care provider. For example, buccal cells can be obtained using a buccal swab or brush, or using mouthwash.

Kits as provided herein can also include a mailer (e.g., a postage paid envelope or mailing pack) that can be used to return the sample for analysis, e.g., to a laboratory. The kit can include one or more containers for the sample, or the sample can be in a standard blood collection vial. The kit can also include one or more of an informed consent form, a test requisition form, and instructions on how to use the kit in a method described herein. Methods for using such kits are also included herein. One or more of the forms (e.g., the test requisition form) and the container holding the sample can be coded, for example, with a bar code for identifying the subject who provided the sample.

### VI. DATABASES AND REPORTS

Also provided herein are databases that include medical information including the genetic and LPS exposure status of a patient. The list is stored, e.g., on a flat file or computer-readable medium. The databases can further include information regarding one or more subjects, e.g., whether a subject is affected or unaffected, clinical information such as endophenotype, age of onset of symptoms, any treatments administered and outcomes (e.g., data relevant to pharmacogenomics, diagnostics or theranostics), and other details, e.g., about the disorder in the subject, or environmental or other genetic factors. The databases can be used to detect correlations between a particular allele or genotype and the information regarding the subject.

The methods described herein can also include the generation of reports, e.g., for use by a patient, care giver, payor, or researcher, that include information regarding a subject's response allele(s), and optionally further information such as treatments administered, treatment history, medical history, predicted response, and actual response. The reports can be recorded in a tangible medium, e.g., a computer-readable disk, a solid state memory device, or an optical storage device.

### VII. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### Materials and Methods

Patients. This prospective case-control study was conducted in Buenos Aires, Argentina between 2003 and 2006. Participating hospitals cared for infants in the Center (at Hospital Francés), West (Hospital Nacional Dr. Alejandro Posadas), and South (Hospital Evita Pueblo de Berazategui, and Hospital Mi Pueblo de Florencio Varela) of the city. These last two regions are separated by 40 highway miles and by other areas of the city. Therefore, the possibility of even small groups of people from either population (who typically lack private transportation) moving to the other district is extremely low.

Previously healthy full term infants younger than one year of age and born after September 15 of the previous year (fifteen days after the end of RSV season in Buenos Aires (Ferolla et al., 2013 and Laham et al., 2004) with signs and symptoms of bronchiolitis for the first time in their lives were invited to participate. Bronchiolitis was defined as a constellation of clinical signs including wheezing with or without cough, rales, dyspnea, and increased respiratory rate and retractions of the respiratory muscles. The diagnosis was performed by trained pediatricians.

Selection of criteria for RSV disease severity was based on a clinically relevant endpoint: previously healthy full-term infants with bronchiolitis were recruited as cases when their oxygen saturation upon enrolment was <93% when breathing room air. Controls were infants with an oxygen saturation 93% while breathing room air. In addition, these criteria allow comparison with numerous previous studies (Tal et al., 2004, Inoue et al., 2007, Puthothu et al., 2006, Paulus et al., 2007 and Kresfelder et al., 2011).

Exclusion criteria included known or suspected impairment of immunological function, major congenital oral malformations, chronic lung disease, cardiac disease, prematurity (gestational age <37 weeks), neuromuscular disorders affecting swallowing, and known or suspected coagulation disorders or bleeding tendency. All Institutional Review Boards approved the protocol, and written and witnessed informed consent was obtained from all parents or guardians.

Epidemiological data reported by parents or guardians and clinical data by physical examination were collected by a pediatrician investigator. Epidemiological information included age, gender, breastfeeding on enrolment, smoking at home, day care attendance, parental history of asthma, and presence of siblings younger than 14 years of age in the household. Clinical information included need for and duration of oxygen supplementation, need for and duration of hospitalization, need for intensive care, and deaths. Investigators monitored the clinical evolution of participating infants through phone calls and/or hospital visits for 7 days after enrolment. Therefore, any change in their clinical status was accounted for in the study (i.e., worsening clinical condition converting a control into a case).

Collection and testing of nasal aspirates. Nasal secretions were obtained from all infants at time of enrollment, by nasal aspiration using 1 ml of sterile saline solution (Analyticals; Chemit). Nasal aspirates (2 ml) were immediately aliquoted, snap frozen in dry ice and transported to the laboratory. Initial diagnosis of RSV infection was performed by direct immunofluorescent assay (Light Biodiagnostic). Results were immediately reported to participating pediatricians. Those infants with positive RSV tests were asked to return for a second visit 5-9 days after enrolment. Aliquots of nasal aspirates were stored at −80° C. for cytokine, mRNA and viral titer determinations. Viral titers were quantified in nasal aspirates using real time PCR. Briefly, RNA was extracted using RNA Easy Minikit (Qiagen, Valencia, Calif.). Real time was perfomed using an RSV Taqman probe with the sequence: 6FAM-CAA TGA TCA TGA TTT ACC TAT TG-MGBNFQ (SEQ ID NO: 1). Viral titers were estimated using a standard curve with known concentrations of RSV.

Collection and Processing of Blood Samples (2003-2006).

Blood was collected from all participating infants at the time of enrolment by venipuncture in the first population (nasal aspirates were used for genotyping in the 2010-2013 population). Blood samples were transferred to the central laboratory and 500 μL stored at −80° C. for genotyping. Serum was stored at −80° C. for IgE determinations. In RSV-infected patients, an additional blood sample was obtained 5-9 days later and peripheral blood mononuclear cells (PBMC) separated by Ficoll-Hypaque (Sigma Aldrich) gradient. 10⁶ PBMC were incubated for 72 h. in culture with 1 μg of immunoaffinity purified protein F and an additional 10⁶ PBMC with 1 μg of immunoaffinity purified protein G. Supernatants and RNA were harvested and stored at −80° C.

Assessment of LPS Environmental Levels.

Samples from cradle and bed sheets from the bedroom of participating infants (2003-2006) were collected in fiberglass filters. LPS was assessed by LAL test in sample dilutions. Results are reported as UI/m (Simoes, J Pediatr, (2003). A similar procedure was used to assess LPS levels in a convenience sample of 120 homes from the 2010-2013 cohort.

Cytokine Determinations.

IL-6, IL-8, IL-1β and TNFα levels were measured in nasal aspirates using a microbead array (Becton Dickinson). IL-4, IFNγ, IL-5, IL-9, IL-13 and IL-17 levels in nasal aspirates were assessed using BioSource ELISA kits following manufacturer's instructions. The same kits were used to assess IL-4 and IFNγ levels in supernatants from PBMC stimulated with RSV G and F. IgE was determined in serum by electrochemoluminiscence (Roche Diagnostics).

GATA3/T-Bet and TLR4 mRNA Expression in Nasal Secretions.

GATA3, T-bet, TLR4, CD14, MD2 and TLR2 mRNA expression were assessed in nasal aspirates of 120 randomly selected subjects from all study groups by real time PCR (Applied Biosystems, Foster City, Calif.). RNA was extracted using the RNA Easy Minikit (Qiagen). cDNA synthesis was performed by Superscript First Strand cDNA Synthesis Assay (Invitrogen) and expression assayed by Taqman. β-actin mRNA expression was used as a housekeeping gene (Applied Biosystem).

Genotyping.

DNA was isolated from whole blood samples using the Gentra Puregene kit (Gentra Systems, Minneapolis, Minn.) and characterized for purity and concentration on a Beckman Coulter DU640 spectrophotometer (Beckman Coulter, Fullerton, Calif.). Low yield samples were whole genome amplified with the Qiagen REPLI-g kit (Qiagen). TLR4 Asp299Gly and Ile399Thr mutations were assessed by allelic discrimination with pre-optimized assays from Applied Biosystems (Applied Biosystems).

Briefly, the TLR4 polymorphic sites were amplified from 10 ng of genomic DNA, 12.5 μl of TaqMan® Genotyping Master Mix, and 1.25 μL of each 20× pre-optimized assay mix; C_{—}11722238 (Asp299Gly, rs4986790) and C_{—}11722237 (Ile399Thr, rs4986791) respectively. Each 20× mix consisted of 18 μM forward and reverse primers and 8 μM of each allele-specific fluorescent-labeled probe. Standard PCR cycling conditions were used, with initial denaturation at 95° C. for 10 minutes, followed by 35 cycles of 92 C for 15 sec and 60° C. for 1 minute. Allele specific PCR products were detected on an ABI 7000 (Applied Biosystems) and clustered by genotype using ABI 7000 sequence detection software. Ambiguous samples were clustered manually and verified by sequencing. Control samples with known genotype, as verified by DNA sequencing, were run with each plate.

CD14 C-159T, CD14 C-550T and IL4 C-590T mutations were analyzed by RFLP. Polymorphic sites were amplified from 50 ng of genomic DNA, 12.5 μL of Failsafe buffer (Epicentre Biotechnologies, Madison, Wis.), 1 μM forward and reverse primer, and 0.25 μL of Taq polymerase enzyme mix (Epicentre Biotechnologies). Resulting PCR products were digested with a specific restriction enzyme (New England Biolabs, Ipswich, Mass.) at 37° C. for 4 hours and run on 3% agarose gels for fragment detection. Control samples with known genotype, as verified by DNA sequencing, were run with each plate. The table lists Failsafe buffers, primer sequences and restriction enzymes used for the RFLP assays.

| Polymorphic | Failsafe | | Restriction |
|---|---|---|---|
| site | buffer | Primer sequence | enzyme |
| CD14 C-159T | G | Forward 5′-ATC ATC CTT TTC CCA CAC C-3′ | Hae III |
| rs2569190 | | (SEQ ID NO: 2) | |
| | | Reverse 5′-AAC TCT TCG GCT GCC TCT-3′ | |
| | | (SEQ ID NO: 3) | |
| CD14 C-550T | H | Forward 5′-GGA AGG GGG AAT TTT TCT TTA GGC-3′ | Hae III |
| rs5744455 | | (SEQ ID NO: 4) | |
| | | Reverse 5′-GGC AGT GTC CTG ATG ACT CAG G-3′ | |
| | | (SEQ ID NO: 5) | |
| IL4 C-590T | F | Forward 5′-TAA ACT TGG GAG AAC ATG GT-3′ | Ava II |
| rs2243250 | | (SEQ ID NO: 6) | |
| | | Reverse 5′-TGG GGA AAG ATA GAG TAA TA-3′ | |
| | | (SEQ ID NO: 7) | |

As a quality control, assays were repeated on 5% of the samples, and results were 100% concordant. In addition, because of potential ethnic diversity in the study population, 33 ethnic-specific genomic markers were evaluated on the Sequenom iPlex platform by Bioserve to rule out admixture.

Second Population (2010-2013).

Infants were recruited using the same criteria as in 2003-2006 from hospitals caring for middle income, insured families (Swiss Medical Center, CEMIC, Hospital Español) in the Central region and public institutions caring for low income families (Hospital Pedro de Elizalde and Hospital Lucio Melendez) in the Southern region. Genotyping was performed in nasal aspirates.

Mice.

Seven-day old C57BL/10 mice and C57BL/10 mice heterozygous for Tlr4^{lps-del} (Tlr4^{+/-} mice) (Jackson Laboratories, ME) were inoculated with 100 μg of LPS or placebo every two days for 14 days and challenged with 10⁶ pfu IN of RSV line 19 at the age 28 days. Unlike RSV A2, line 19 induces dose-dependent AHR in mice (Lukacs et al., 2006). Four-week old BALB/c (wt, Stat 6^{-/-} and Stat 1^{-/-}) mice and tamoxifen-treated C57BL/6 wt and C57BL/6 mice conditionally deficient on GATA3 (GATA3^{fl/fl}) were infected with RSV line 19 nine days later for evaluation of the role of transcription factors in RSV bronchiolitis.

AHR to increasing concentrations of metacholine (mCh) was determined as previously described (Delgado et al., 2009). Briefly, five days after challenge, the inventors anesthetized the mice with a mix of ketamine (100 mg/kg) and xilazine (10 mg/kg) intraperitoneal, intubated them and ventilated them at a rate of 120 breaths per min with a constant tidal volume of air (0.2 ml). The inventors then paralyzed the mice with decamethonium bromide (25 μg/kg) and, after establishing a stable airway pressure, determined airways resistance to aerosolized methacholine (0.01-30 mg/kg). Immunoassays for inflammatory cytokines in BAL fluids were determined using kits from Biosource, following the manufacturer's instructions. All studies were approved by the ACUC.

Statistical Analysis.

Data were analyzed using the Chi Square test for proportions and Student's T or Mann Whitney tests for continuous variables where appropriate. The role of epidemiologic and clinical variables as independent predictors of severe disease was examined by univariate and multivariable logistic regression. The Hardy Weinberg equilibrium was calculated for every SNP examined. Statistical analyses were performed using a Stata 11.2 package for IBM-PC (Stata Corp, College Station, Tex.). A p value <0.05 was considered significant.

### Example 2

### Results

Infants with RSV Bronchiolitis are Exposed to Different Levels of Environmental LPS.

Seven hundred and sixty eight infants with bronchiolitis (426 with severe disease and 342 with mild disease) were prospectively recruited during the winter seasons of 2003-2006 in the emergency rooms (ER) and outpatient clinics (OPC) of participating institutions during the inventors' first study. Fifty-four (7%) parents declined their infant's participation; these infants had similar characteristics as those enrolled in the study (not shown).

RSV was detected in nasal secretions of 418 (54%) participating patients. Severe RSV bronchiolitis was found in 246 cases (59%), while 172 (41%) infants had mild RSV bronchiolitis, and served as controls.

Infants from three geographical regions of Buenos Aires participated in the first study. Those from the western and southern regions of the Buenos Aires district lived with families of low socioeconomic status (Supp. Table 2). Variables reported to affect the risk of severe RSV bronchiolitis (Collins et al., 2001 and Simoes, 2003) were similar in infants from the west and south low-income regions (Supp. Table 3).

The third group of infants lived in middle class families (central region; Supp. Table 2). In a comparison of the frequency of risk factors between low and middle-income groups, exposure to cigarette smoke (Larsson et al., 2008) was observed more often in low-income than in middle-income families (Supp. Table 3); but only age and number of siblings <14 years of age at home influenced severity of RSV bronchiolitis (Supp. Table 4). No differences were detected in duration of symptoms and oxygen requirement, or need for intensive care between the RSV-infected populations in low and high-income groups (Supp. Table 5).

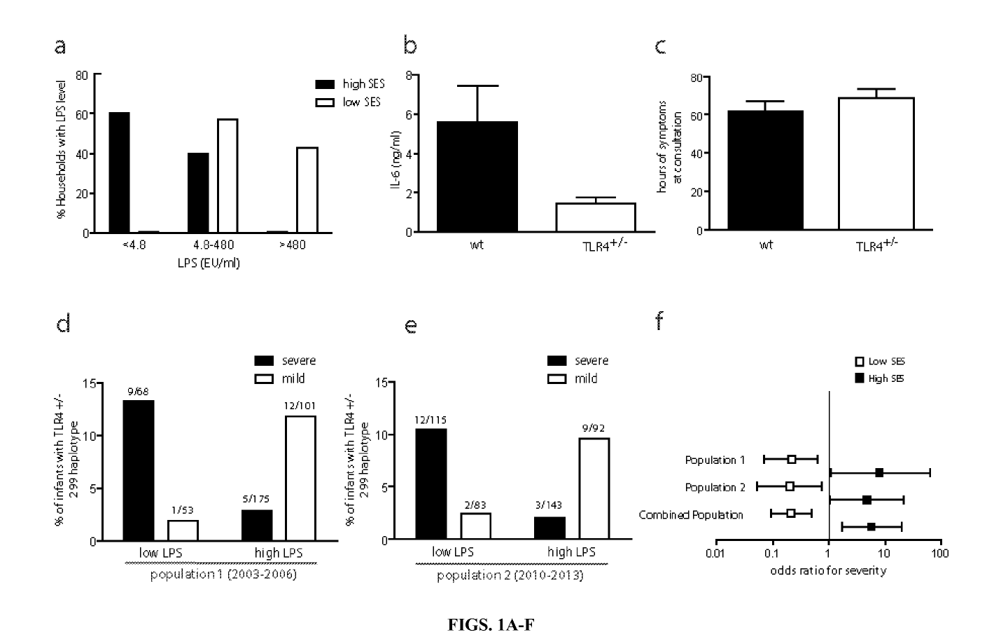

Interestingly, socioeconomic status was associated with differences in LPS levels in infant bedrooms (FIG. 1A). Homes in low socioeconomic regions had similar levels of bedroom LPS (p=0.89), which was significantly higher than those in neighborhoods of high socioeconomic status (p<0.01).

TLR4 Asp299Gly and Thr399Ile Alleles Modulate IL-6 and IL-8 In Vivo in RSV-Infected Infants.

The cohort was genotyped for the Asp299Gly and Thr399Ile alleles (Supp. Table 6). The overall distributions of the two alleles were in Hardy-Weinberg equilibrium, and frequencies were similar to those published for other populations (Ferwerda et al., 2007). Thirty-three ethnic-specific genomic markers revealed neither evidence of admixture nor significant ethnic differences between the groups (Supp. Table 7).

Comparison of interleukin-6 (IL-6) and IL-8 levels in respiratory secretions of RSV-infected infants with two major TLR4 alleles and those with one TLR4D299G allele showed significantly lower cytokine levels in secretions of infants with a minor allele (FIG. 1B for IL-6). No difference in time from initiation of symptoms to sampling was observed (FIG. 1C). Individuals homozygous for the SNP were too infrequent for statistical analyses. Identical analyses were performed with the Thr399Ile allele and the Asp299Gly/Thr399Ile haplotype. Results were similar to those with the Asp299Gly allele alone. Therefore, hereafter only analyses with the Asp299Gly SNP are presented.

The Interaction Between TLR4 Genotype and the Environment is Associated with Severity of RSV Bronchiolitis.

The TLR4D299G allele was present in 27/397 (6.8%) of RSV-infected infants and 21/331 (6.3%) of infants infected with other agents (p=0.9; Supp. Table 6).

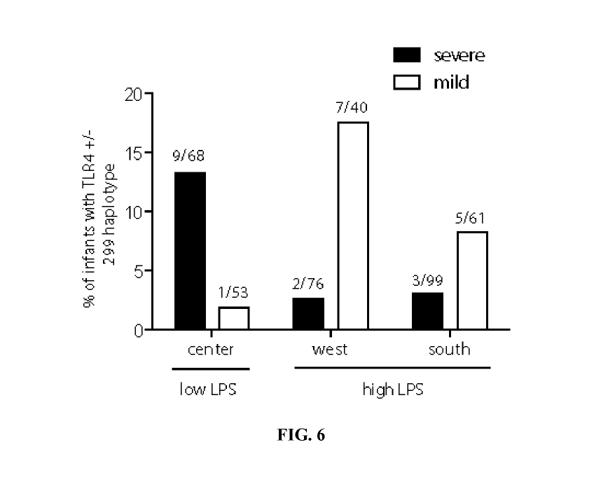

In infants from the western and southern regions, characterized by environments with high LPS levels, Asp299Gly was more frequent in children with mild RSV bronchiolitis (FIG. 6). In fact, both low income groups had similar risk factors (Supp. Table 3), additional indicators of disease severity (Supp. Table 5) and distribution of TLR4 genotypes (p=0.3), and were therefore subsequently analyzed as one (FIG. 1D). Conversely, in infants from regions with low LPS exposure, Asp299Gly was more frequent in patients with severe disease (FIG. 1D). The impact on severity of illness of the interaction between TLR4 and environments with different LPS levels was significant, even after adjusting for risk factors affecting severity of RSV bronchiolitis (Table 1) (Collins et al., 2001 and Simoes, 2003). TLR4 genotypes did not affect severity of illness in patients with non-RSV bronchiolitis, suggesting that the gene-environment interaction is virus-specific (Table 1).

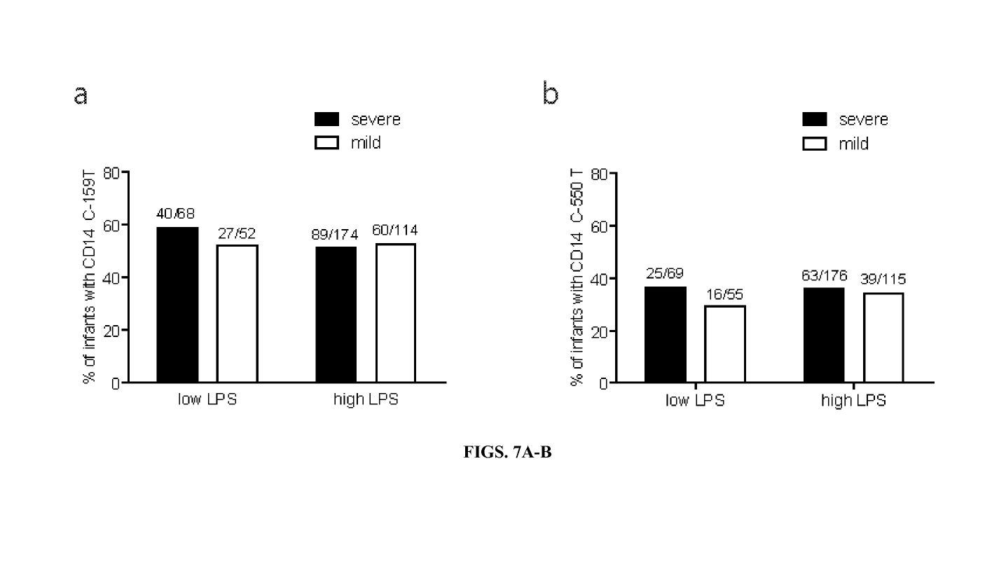

CD14 is a component of the CD14/TLR4 receptor that enhances TLR4 responses (Simpson et al., 2006 and Miller et al., 2005). To determine whether CD14 affected RSV disease severity, the inventors asked whether the CD14 C-159T and CD14 C-550T SNPs also associated with RSV bronchiolitis (FIGS. 7A-C; Supp. Table 8). Allele frequencies for both SNPs were in Hardy-Weinberg equilibrium, and did not differ from those reported previously^{15, 16, 25}. Unlike TLR4 Asp299Gly, CD14 C-159T and CD14 C-550T did not affect RSV disease severity.

Role of RSV Titer in Disease Severity.

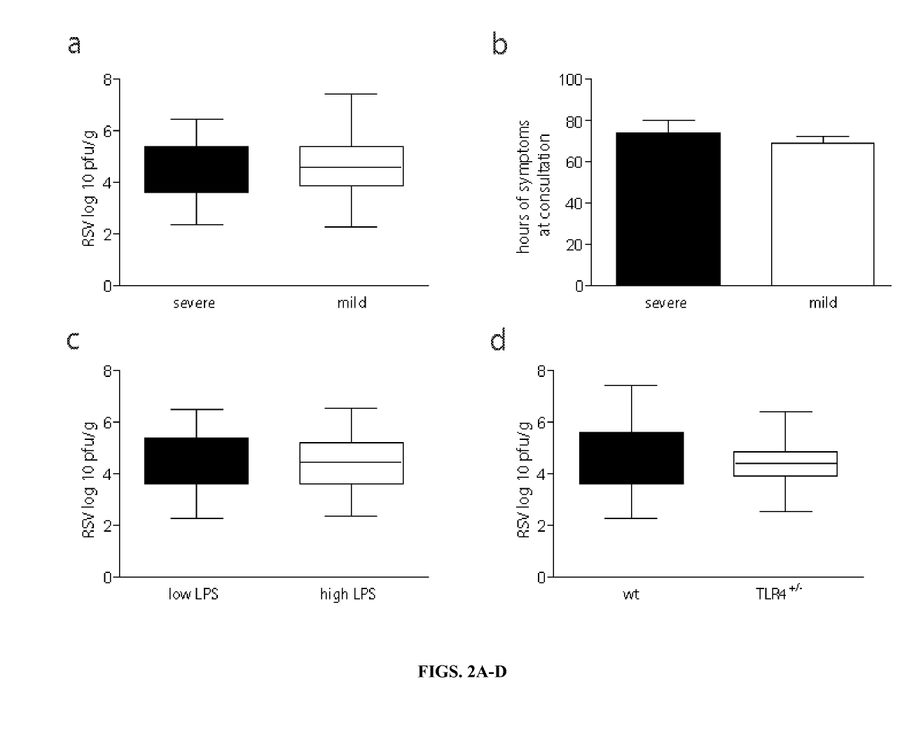

To explore candidate variables that may affect severity of RSV bronchiolitis, the inventors first compared RSV titers in respiratory secretions. Comparison between cases and controls revealed no differences (FIG. 2A). Time from initiation of symptoms to sampling was similar between groups (FIG. 2B). Neither differential LPS exposure (FIG. 2C) or different genotypes (FIG. 2D) affected RSV titers. These observations do not support an association between severity of RSV disease and virus titer in respiratory secretions.

RSV-Mediated Inflammation in the Lungs is Determined by Environmental Exposures.

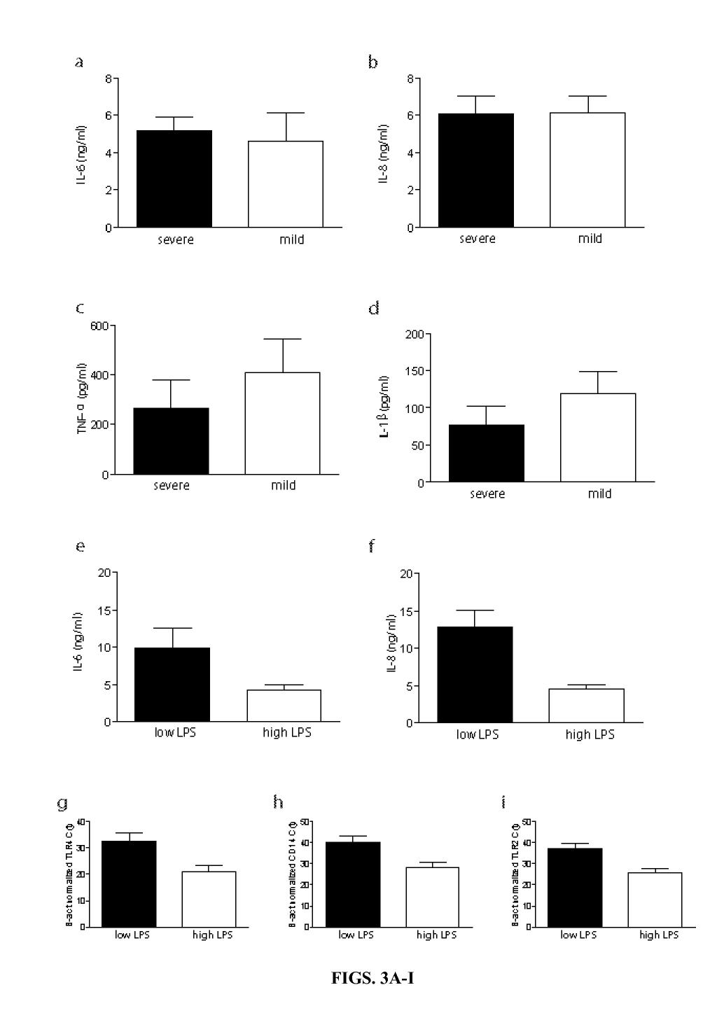

The inventors then examined whether severity of RSV bronchiolitis was associated with inflammation in the respiratory tract. For this purpose, the inventors measured levels of interleukin-6 (IL-6), IL-8, tumor necrosis factor-α (TNFα) and IL-1β in respiratory secretions of RSV-infected infants and found no differences in levels between mildly and severely-ill subjects (FIGS. 3A-D).

Interestingly, inflammatory cytokines were more abundant in secretions of patients living in areas of low compared to high chronic LPS exposure (FIGS. 3E-F). These findings suggest that environment rich in LPS influence the level of cytokine production during RSV infection. To explore whether the different inflammatory responses in these populations were associated with differential expression of pattern recognition receptors (PRR) in the respiratory tract before infection, the inventors compared TLR4 expression in asymptomatic infants residing in the same neighborhoods as those infected with RSV during the study. Expression of TLR4 in the respiratory tract of asymptomatic infants from neighborhoods with high environmental LPS levels was suppressed compared to infants from neighborhoods with low LPS levels (FIG. 3G). The inventors reasoned that other PRR might also be modulated by environmental exposures. Indeed, CD14, TLR2 and MD2 mRNA levels were down-regulated in infants chronically exposed to LPS (FIGS. 3H-I and not shown) (Perros et al., 2011).

The TLR4-Environment Interaction Modulates GATA3/T-Bet Ratios Affecting RSV Disease Severity.

In mice, exposure to high levels of pathogen-associated molecular patterns (PAMP) promotes Th1 bias and low levels promote Th2 responses through PRR (Eisenbarth et al., 2002). The inventors therefore reasoned that the population of poor infants with low PRR expression would have “weak” PRR-RSV interactions (equivalent to a low inoculum of a PAMP) and bias the immune response to Th2, while middle-class infants with high PRR expression would bias the response against RSV to Th1.

To explore this hypothesis, the inventors examined in both populations expression of the Th2 and Th1 transcription factors GATA3 and T-bet (Glimcher, 2007 and Zhu et al., 2006). They hypothesized that the ratio between these master regulators during RSV infection would be affected by the interaction between TLR4 and the environment, and play a role in severity of illness.

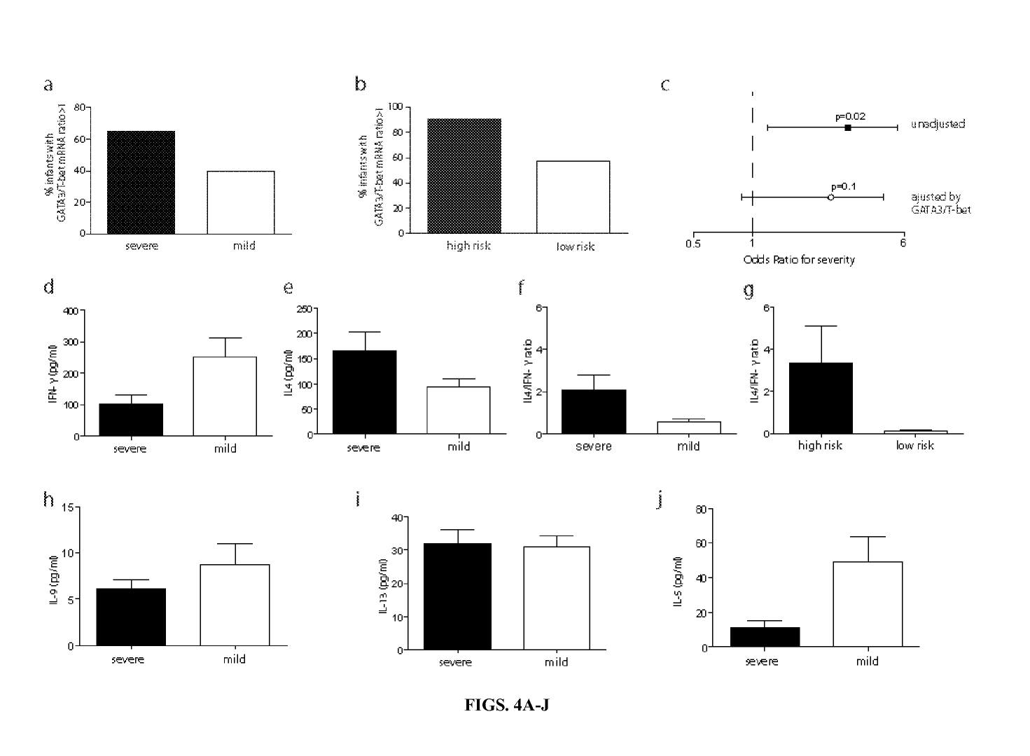

Interestingly, in the subgroup of infants with available measurements (n=120), Th2 GATA3/T-bet ratios >1 were associated with RSV disease severity (FIG. 4A; OR=2.82 [1.14-6.96]; p=0.02). Furthermore, infants at high risk for severe disease (low LPS exposure and a TLR4D299G allele+high LPS exposure and two major alleles) had a higher frequency of GATA3/T-bet ratios >1 than low risk infants (FIG. 4B; OR=7.61 [2.83-20.4]; p<0.0001). Finally, the effect of the TLR4-environment interaction on severe RSV bronchiolitis (OR=2.57 [1.19-5.55]; p=0.02) was weakened by adjusting for GATA3/T-bet (OR=2.03 [0.88-4.69]; p=0.1), even after subsequently adjusting for RSV risk factors (p=0.1; FIG. 4C). These findings suggest that the GATA3/T-bet ratio is involved in the LPS/TLR4 pathway to severe RSV bronchiolitis.

Low Interferon-γ (IFNγ) and High IL-4 Levels in Respiratory Secretions of Infants with Severe RSV Bronchiolitis.

The inventors therefore investigated the role of Th1 (IFNγ) and Th2 (IL-4, IL-5, IL-9 and IL-13) cytokines in bronchiolitis severity. IFNγ levels were lower in infants with severe compared to mild illness (FIG. 4D). Conversely, IL-4 levels were higher in infants with severe RSV infection (FIG. 4E). In fact, IL-4/IFNγ ratios were also significantly higher in severely ill and at risk infants, confirming the inventors' observations (FIGS. 4F-G). The inventors detected no differences in IL-9 and IL-13 levels in respiratory secretions between mildly and severely ill patients (FIGS. 4H-I). Interestingly, IL-5 levels associated with protection (FIG. 4J). IL-17 was undetectable in all infants.

RSV Bronchiolitis and Systemic Th2 Bias.

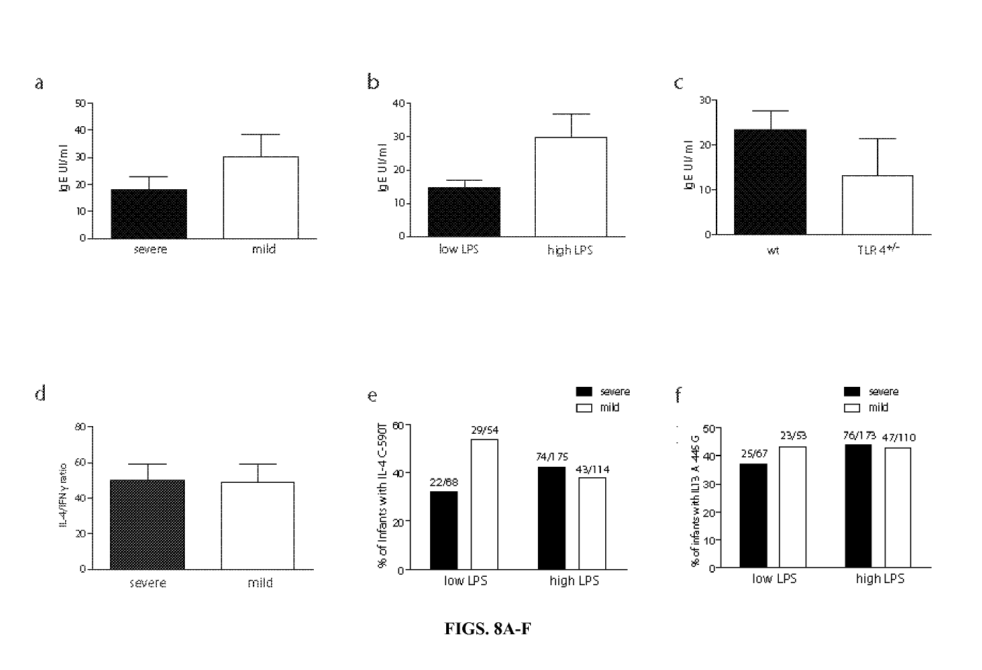

The inventors then compared total serum IgE levels and IL-4/interferon-γ (IFNγ) ratios in peripheral blood of infants with severe and mild RSV disease and observed no differences between groups (FIGS. 8A-D). Finally, neither the distribution of gain-of-function IL4 SNPs (C-590T) nor IL13 SNPs (A-445G) associated with severe disease in this population (FIGS. 8E-Ff; Supp. Table 9).

TLR4 Genotype and Environment in a Second Infant Population.

To confirm their observations, the inventors studied a second population of 433 infants with RSV LRI between 2010 and 2013. These infants attended different hospitals than those in the first cohort in the same Central and Southern regions (FIGS. 1E-F; Supp. Table 10). 235 infants lived in low-income homes in the Southern region characterized by high levels of environmental LPS, while 198 infants lived in an urban, middle class, low LPS environment in the Central region (Supp. Table 10). In this case, the Asp299Gly allele was detected in 12 infants from the group of homes in the Southern region versus 14 from the Central region, for an overall rate of 6.0% (p=0.64 vs. first population). Again, rates of the Asp299Gly allele were higher in severe cases among infants from the group of high-income, urban low LPS environments (OR=4.71[1.03-21.68]) and in mild cases among those in low-income, high LPS environments (OR=0.20[0.05-0.75]; FIG. 1E). Moreover, the impact of the TLR4-environmental interaction on disease severity was significant when analyzing this second population (p=0.002) and both populations together (p<0.001; FIG. 1F).

A Mechanistic Role for Th2 Bias in a Mouse Model of RSV Bronchiolitis.

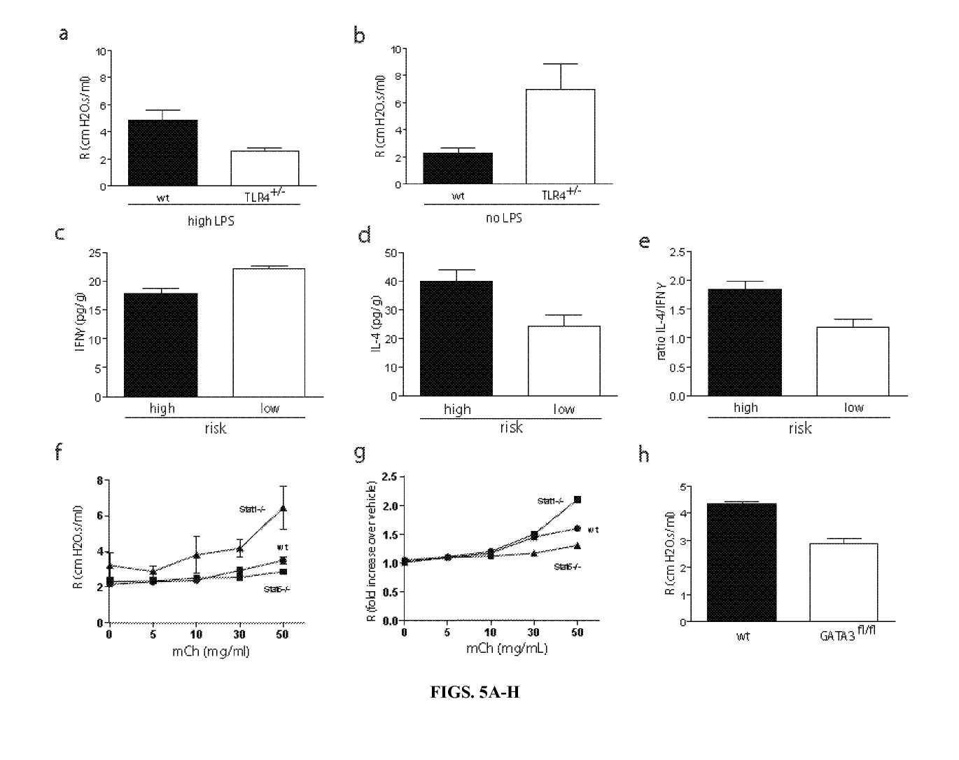

Finally, the inventors investigated whether a Th bias played a mechanistic role in RSV disease severity in mice. For this purpose, they evaluated airways hyperreactivity (AHR) in rodents infected with RSV. First, they replicated the gene-environment interaction observed in infants with RSV bronchiolitis (FIGS. 5A-B). Pre-exposure of C57BL/10 and Tlr4^{+/-} mice to a chronic high dose of LPS (mimicking infants with low SES) followed by RSV intranasal inoculation led to enhanced AHR in wt compared to Tlr4^{+/-} animals (FIG. 5A). Conversely, no LPS (“high SES mice”) followed by RSV resulted in increased AHR in Tlr4^{+/-} compared to wt mice (FIG. 5B). As expected, no differences in viral pulmonary titers or inflammation, as determined by IL-6 and IL-1β immunoasays in BAL fluids, were observed between groups of different disease severity (not shown).

Following these results and the observations in infants, the inventors compared IFNγ and IL-4 levels in respiratory secretions of mice at “high” vs. “low” risk for severe disease. As described in infants, at risk mice had higher IL-4, IFNγ and IL-4/IFNγ ratios in the respiratory tract (FIG. 5C-E).

Finally, the inventors tested the role of Th2 bias in disease severity using Stat-1^{-/-} and Stat-6^{-/-} mice, and mice conditionally deficient on GATA3 (GATA3^{fl/fl}) (FIGS. 5F-H). T-bet activation is directly associated with prior Stat-1 activation (Robinson and O'Garra, 2002). Induction of Gata3 in naive CD4⁺ T cells requires Stat-6 activation (Kaplan et al., 1996). Five days after RSV inoculation, Stat-6^{-/-} mice had lower AHR than WT controls. Stat-1^{-/-} mice had higher AHR than both other groups (FIGS. 5F-G). The role of GATA3 was confirmed in wt and GATA3^{fl/fl}mice (FIG. 5H). Five days after RSV infection, GATA3^{fl/fl} mice had lower AHR than wt mice, favoring a Th2 bias in pathogenesis of RSV disease.

TABLE 1
Odds ratio for severe bronchiolitis: association with TLR4 mutation according to the environment

| | | | Crude | | | Adjusted† | | |
|---|---|---|---|---|---|---|---|---|
| Environment | TLR4 Mutation | n/N (%) | OR (CI95%) | p* | p** | OR (CI95%) | p* | p** |
| Low LPS^{#} | Yes | 9/10 (90.00) | 7.93 (0.97-64.73) | 0.053 | | 11.18 (1.25-100.20) | 0.031 | |
| | No | 59/111 (53.15) | 1.00 | | | 1.00 | | |
| | | | | | 0.0003 | | | 0.0003 |
| High LPS^{#} | Yes | 5/17 (29.41) | 0.22 (0.08-0.64) | 0.005 | | 0.21 (0.07-0.63) | 0.006 | |
| | No | 170/259 (65.64) | 1.00 | | | 1.00 | | |
| Low LPS^{##} | Yes | 5/8 (62.50) | 1.49 (0.33-6.87) | 0.606 | | 1.51 (0.27-8.43) | 0.634 | |
| | No | 29/55 (52.73) | 1.00 | | | 1.00 | | |
| | | | | | 0.511 | | | 0.455 |
| High LPS^{##} | Yes | 6/13 (46.15) | 0.79 (0.26-2.42) | 0.684 | | 0.81 (0.25-2.61) | 0.721 | |
| | No | 133/256 (51.95) | 1.00 | | | 1.00 | | |

†Adjusted by age, sex, breastfeeding, number of siblings and smoking at home
*Significance of OR for severe disease
**Significance for interaction between TLR4 mutation and environment
^{#}RSV Positive
^{##}RSV Negative

SUPPLEMENTARY TABLE 1
Genotype frequencies for TLR4 SNPs in previous studies of infants
with RSV bronchiolitis.

| | | | | % Mutations | | | |
|---|---|---|---|---|---|---|---|
| Manuscript (ref.) | Country | TLR4 SNP | n | Cases | Controls | OR | p |
| Tal G. et. al. JID 2004 (14). | Israel | Both | 181 | 20 | 4 | 4.9 (1.6-15.3) | 0.003 |
| Paulus S. C. et. al. Clin Immunol 2007 (17). | Canada | 299 | 342 | 5 | 10 | NA | ns |
| Puthothu B. Dis Markers 2006 (16). | Germany | 299 | 401 | 4 | 8 | NA | 0.05 |
| Kresfelder T. L. et. al. J Med Virol 2011 (18). | South Africa | Both | 409 | 6 | 12 | 0.48 (0.23-0.99) | — |
| Inove Y. et. al. JID 2007 (15). | Japan | Both | 104 | 0 | 0 | NA | — |

SUPPLEMENTARY TABLE 2
Socioeconomic status in West, South and Central
communities in Buenos Aires (2003-2006)

| | low SES‡ | high SES |
|---|---|---|
| Under poverty line* (%) | 60 | 0 |
| Crowding** (%) | 45 | 3 |
| Well water (%) | 38 | 0 |
| No sewage (%) | 62 | 0 |
| Incomplete elementary school (%) | 47 | 1 |

*income less than U$S 161/month
**more than three people per room
‡West + South regions
From
National Institute of Statistics and Census, Ministry of Economy, Argentina. Censo nacional de población, hogares y viviendas. 2001 Hospital Posadas and Hospital Evita Pueblo epidemiological records.

SUPPLEMENTARY TABLE 3
Demographic characteristics of infants in the first population.

| | RSV positives (n = 418) | | | | | RSV negatives (n = 350) | | | | | |
|---|---|---|---|---|---|---|---|---|---|---|---|
| Region | West | South | p* | Center | p** | West | South | p* | Center | p** | p*** |
| N | 122 | 171 | | 125 | | 152 | 131 | | 67 | | |
| Males (%) | 66(54) | 87(51) | 0.58 | 75(62) | 0.17 | 86(57) | 75(57) | 0.9 | 42(63) | 0.46 | 0.34 |
| Breastfed (%) | 98(80) | 138(80) | 0.93 | 109(87) | 0.13 | 123(81) | 101(77) | 0.43 | 50(75) | 0.52 | 0.14 |
| Smokers at home (%) | 73(60) | 116(68) | 0.16 | 55(44) | <0.001 | 87(57) | 82(62) | 0.36 | 21(31) | 0.001 | 0.25 |
| Daycare (%) | 4(3) | 2(1) | 0.24 | 9(7) | 0.02 | 7(5) | 3(2) | 0.28 | 10(15) | 0.05 | 0.16 |
| Sib <14 y (med) | 1 | 1 | 0.53 | 1 | 0.31 | 1 | 2 | 0.16 | 1 | 0.19 | 0.60 |
| Age (mo, mean) | 4.3 ± 2.8 | 3.6 ± 2.2 | 0.1 | 4.2 ± 2.5 | 0.41 | 5.2 ± 2.1 | 4.0 ± 2.3 | 0.001 | 5.3 ± 2.1 | 0.06 | 0.03 |

*West vs. South
**West + South vs. Center
***RSV positives vs. RSV negatives

SUPPLEMENTARY TABLE 5
Additional indicators of severity in infants with REV bronchiolitis.

| Region | West | South | Center |
|---|---|---|---|
| Number of infants | 78 | 99 | 69 |
| Oxygen supplementation (d; mean ± SD) | 5 ± 3 | 6 ± 2 | 6 ± 3 |
| Hospitalization (d; mean ± SD) | 6 ± 2 | 7 ± 2 | 7 ± 3 |
| Need for pediatric intensive care (n) | 1 | 2 | 4 |
| Deaths (n) | 0 | 0 | 0 |

Note:
No significant differences (p < 0.05) observed between groups.

SUPPLEMENTARY TABLE 4
Epidemiological risk factors for severe RSV bronchiolitis.

| | | | | | All infants | | | |
|---|---|---|---|---|---|---|---|---|
| | low LPS | | high LPS | | univariate | | multivariate | |
| | RSV positives | | | | | | | |
| n | 125 | | 293 | | 418 | | 418 | |
| Males OR (95% CI) | 0.66 | (0.29-1.49) | 0.89 | (0.54-1.46) | 0.83 | (0.55-1.26) | 0.84 | (0.55-1.26) |
| Breastfeeding OR (95% CI) | 3.22 | (0.97-10.69) | 0.68 | (0.36-1.29) | 0.96 | (0.56-1.64) | 0.96 | (0.56-1.65) |
| Smokers OR (95% CI) | 0.72 | (0.33-1.59) | 1.32 | (0.79-2.19) | 1.10 | (0.73-1.67) | 1.10 | (0.72-1.67) |
| Daycare OR (95% CI) | 0.5 | (0.11-2.27) | 0.23 | (0.04-1.43) | 0.35 | (0.11-1.1) | 0.35 | (0.11-1.13) |
| Siblings <14 y OR (95% CI) | 1.5* | (1.01-2.23) | 1.14 | (0.97-1.34) | 1.2* | (1.04-1.39) | 1.2* | (1.03-1.39) |
| Age (months) OR (95% CI) | 0.81* | (0.69-0.96) | 0.82* | (0.74-0.90 | 0.82* | (0.75-0.89) | 0.82* | (0.75-0.89) |
| | RSV negatives | | | | | | | |
| n | 67 | | 283 | | 305 | | 350 | |
| Males OR (95% CI) | 1.14 | (0.39-3.33) | 0.92 | (0.56-1.52) | 0.91 | (0.59-1.42) | 0.91 | (0.58-2.98) |
| Breastfeeding OR (95% CI) | 0.70 | (0.19-2.65) | 0.68 | (0.37-1.27) | 0.69 | (0.4-1.19) | 0.71 | (0.41-1.22) |
| Smokers OR (95% CI) | 2.66 | (0.72-9.89) | 1.47 | (0.89-2.44) | 1.34 | (0.86-2.08) | 1.46 | (0.93-2.30) |
| Daycare OR (95% CI) | 1.17 | (0.25-5.4) | 0.38 | (0.09-1.66) | 1.10 | (0.42-2.89) | 0.94 | (0.35-2.51) |
| Siblings <14 y OR (95% CI) | 1.19 | (0.84-1.69) | 1.36* | (1.13-1.63) | 1.30* | (1.11-1.52) | 1.32* | (1.12-1.55) |
| Age (months) OR (95% CI) | 1.12 | (0.93-1.34) | 0.85* | (0.77-0.94) | 0.91* | (0.84-0.99) | 0.91* | (0.84-0.99) |

*p < 0.05

SUPPLEMENTARY TABLE 6
Genotype frequencies for TLR4 SNPs in infants
with bronchiolitis (2003-2006).

| | TLR4 299* | | | | TLR4 399** | | |
|---|---|---|---|---|---|---|---|
| | RSV positives | RSV negatives | Total | | RSV positives | RSV negatives | Total |
| SNP | n (%) | n (%) | n | SNP | n (%) | n (%) | n |
| CC | 369 (92.95) | 310 (93.66) | 679 | AA | 372 (92.77) | 317 (94.07) | 689 |
| CT | 27 (6.80) | 21 (6.34) | 48 | AG | 28 (6.98) | 20 (5.93) | 48 |
| TT | 1 (0.25) | 0 | 1 | GG | 1 (0.25) | 0 | 1 |
| Total | 397 (100) | 331 (100) | 728 | Total | 401 (100) | 337 (100) | 738 |

*sequence failed in 40 for TLR4 299.
**sequence failed in 30 for TLR4 399.

SUPPLEMENTARY TABLE 8
Genotype frequencies for CD14 SNPs
in infants with bronchiolitis (2003-2006).

| | CD14 159* | | | | CD14 550** | | |
|---|---|---|---|---|---|---|---|
| | RSV positives | RSV negatives | Total | | RSV positives | RSV negatives | Total |
| SNP | n (%) | n (%) | n | SNP | n (%) | n (%) | n |
| CC | 107 (26.62) | 111 (32.65) | 679 | CC | 216 (54.82) | 199 (61.04) | 415 |
| CT | 216 (53.73) | 161 (47.35) | 48 | CT | 143 (36.29) | 107 (32.83) | 250 |
| TT | 79 (19.65) | 68 (20.00) | 1 | TT | 35 (8.88) | 20 (6.13) | 55 |
| Total | 402 (100) | 340 (100) | 742 | Total | 394 (100) | 326 (100) | 720 |

*sequence failed in 26 for CD14 159.
**sequence failed in 48 for CD14 550.

SUPPLEMENTARY TABLE 7
Ethnic-specific genomic markers to evaluate population admixture
and reported relative frecuencies in different ethnicities.

| | | | Major elleta frequency | | | |
|---|---|---|---|---|---|---|
| Chromosome ionation | rs Number | SNP | Nat Am | European | African | Hispanics |
| 20q11.22 | rs16434 | Device | 0.56 | 0.65 | 0.17 | 5.51 |
| 1q23.2 | rs2814776 | A/G | 0.99 | 0.99 | 0 | 6.64 |
| 1q42 | rs2752 | G/T | 0.33 | 0.46 | 0.09 | |
| 2p16.1 | rs3287 | A/G | 0.79 | 0.8 | 0.27 | 0.68 |
| 7p22.3 | rs2763 | C/G | 0.48 | 0.84 | 0.86 | 0.72 |
| 9q21.31 | rs2695 | A/G | 0.78 | 0.14 | 0.19 | 5.44 |
| 11q11 | rs1042602 | A/C | 0.05 | 0.47 | 0 | 8.28 |
| 15q14 | rs2862 | C/T | 0.69 | 0.17 | 0.38 | 0.4 |
| 16q212 | rs4646 | A/C | 0.72 | 0.29 | 0.32 | 0.42 |
| 17p12 | rs2516 | C/T | 0.92 | 0.51 | 1 | 0.67 |
| 1q32.3 | rs23632 | G/C | 0.33 | 0.08 | 0.9 | 0.27 |
| 3q22.3 | rs584069 | A/C | 0.53 | 0.86 | 0.51 | 0.73 |
| 6p13.2 | rs830072 | C/T | 0.45 | 0.1 | 0.96 | 0.34 |
| 8p21.3 | rs286 | C/T | 0.45 | 0.1 | 0.96 | 0.34 |
| 9q33.3 | rs516116 | A/G | 0.42 | 0.33 | 0.87 | 0.5 |
| 11q23.1 | rs1079508 | C/T | 0.63 | 0.14 | 0.06 | 0.26 |
| 11q23.1 | rs1809438 | C/T | 0.91 | 0.35 | 0.86 | 0.6 |
| 14q32.12 | rs739394 | C/T | 0.99 | 0.74 | 0.62 | 0.79 |
| 11q23.1 | rs1800404 | A/G | 0.48 | 0.72 | 0.14 | 0.64 |
| 17q21.33 | rs203096 | G/T | 0.28 | 0.72 | 0.65 | 0.69 |
| 16q24.3 | rs2228478 | A/G | 0.96 | 0.56 | 0.49 | 0.88 |
| 1q32.1 | rs10600899 | A/G | | 0.067 | 0.017 | |
| 7q22.1 | rs776745 | A/C | | 0.867 | 0.083 | |
| 7p14.3 | rs13226969 | C/G | | 0.1 | 0.75 | |
| 21q21.1 | rs1035461 | A/G | | 0.488 | 0.561 | |
| 17p13.2 | rs2391 | A/G | 0.67 | 0.49 | 0.98 | 0.56 |
| 5q23.1 | rs3317 | A/G | 0.73 | 0.69 | 0.05 | 0.54 |
| 5q33.2 | rs3340 | A/G | 0.35 | 0.81 | 0.94 | 0.6 |
| 10q23.1 | rs235936 | C/T | 0.37 | 0.49 | 0.38 | 0.42 |
| 7q22.1 | rs2161 | A/G | 0.62 | 0.3 | 0.44 | 0.36 |
| 7p14.3 | rs1655080 | A/G | 0.03 | 0.36 | 0.9 | 0.3 |
| 1q32.1 | rs2055160 | C/T | 0.63 | 0.08 | 0.5 | 0.63 |
| 19q13.42 | rs1989486 | C/T | 0.4 | 0.68 | 0.04 | 5.51 |

From Hoggart CJ, et al. Am J Hum Genet 72 1492-1504 (2003).

SUPPLEMENTARY TABLE 9
Genotype frequencies for IL-13 A-445G and IL-4 C-590T
in infants with bronchiolitis (2003-2006).

| | IL-13 445* | | | | IL-4 590** | | |
|---|---|---|---|---|---|---|---|
| | RSV positives | RSV negatives | Total | | RSV positives | RSV negatives | Total |
| SNP | n (%) | n (%) | n | SNP | n (%) | n (%) | n |
| GG | 167 (41.44) | 135 (39.94) | 302 | CC | 183 (44.53) | 148 (43.27) | 331 |
| AG | 171 (42.43) | 153 (45.27) | 324 | CT | 168 (40.88) | 157 (45.91) | 325 |
| AA | 65 (16.13) | 50 (14.79) | 115 | TT | 59 (14.36) | 36 (10.53) | 95 |
| Total | 403 (100) | 338 (100) | 741 | Total | 410 (100) | 341 (100) | 751 |

*sequence failed in 9 from 750 for IL-13 445.
**sequence failed in 17 for IL-4 590.

SUPPLEMENTARY TABLE 10
Socioeconomic status in low and high income
groups in Buenos Aires (2010-2013).

| | low SES‡ | high SES |
|---|---|---|
| House material* (%) | 25 | 0 |
| Crowding** (%) | 59 | 5 |
| Well water (%) | 30 | 0 |
| No sewage (%) | 62 | 0 |
| Incomplete elementary school (%) | 12 | 0 |

*house material % of tin/mud
**more than three people per room
‡West + South regions
From
Latinoamerican Foundation of Economics Research. Valorización de la canasta básica alimentaria y canasta básica total 2010

### Example 3

### Discussion

This study identifies an important and specific pathogenic mechanism for severe RSV bronchiolitis. The TLR4-environment interaction modulates GATA3/T-bet ratios during RSV infection, and ratios affecting a Th2 bias with high levels of IL-4 and low levels of IFNγ are associated with increased disease severity in infants. The role of these transcription factors and cytokines was confirmed in mice.

This study, in agreement with previous observations (Wright, et al., 2002), does not find evidence supporting a causative role for virus titer in disease severity. The poor record of corticosteroids against RSV (Corneli, et al., 2007), studies reporting low levels of inflammatory cytokines during severe illness (Sheeran et al., 1999), absent inflammation in fatal RSV cases (Welliver et al., 2007), and discrepant inflammatory responses in severe bronchiolitis caused by different viruses (Laham et al., 2004) suggest that an augmented production of inflammatory cytokines may not explain severe bronchiolitis. In this study, severe RSV bronchiolitis correlated with a predominance of GATA3 over T-bet and IL-4 over IFNγ in the respiratory tract (Legg et al., 2003). Previous studies associated IL-9 and IL-13 with severe disease8,10, but this study not replicate those observations. Ethnic, demographic, and/or study design differences may explain these discrepancies.

The role of TLR4 in RSV disease has been a source of controversy. Environmental exposures (Tal et al., 2004 and Kresfelder et al., 2011), lack of power (Puthothu et al., 2006, Paulus et al., 2007 and Kresfelder et al., 2011), choice of controls (Tal et al., 2004, Inoue et al., 2007, Puthothu et al., 2006, Paulus et al., 2007 and Kresfelder et al., 2011), or specific genetic or demographic characteristics of the population (Inoue et al., 2007) may explain differences between human studies. In fact, the inventors' observations and those of others (Marr and Turvey, 2012) suggest that the effect of TLR4 in disease severity does not occur through interaction of this PRR with the virus, but through environmental TLR4 activation conditioning the immune response to infection.

Importantly, this study identifies a group of full term infants without obvious phenotypic characteristics that may be highly susceptible to RSV: TLR4+/− infants from environments with low levels of LPS. Over 80% of them were hospitalized when visiting the ER or OPC with respiratory symptoms due to RSV14. Even if only 7-10% of all TLR4+/− infants visit the ER with RSV infections every year (Hall et al., 2009), hospitalization rates for them would approach those of extreme premature babies (Palivizumab, 1998). But while premature babies <1,500 g at birth are ˜1.5% of the population in western, industrialized societies, TLR4+/− term infants represent ˜10%24. Moreover, 89.5% of North American premature infants hospitalized with RSV LRI in earlier studies were found to be TLR4^{+/-}, while the rate of heterozygosity in the general population was 10.5% (Awomoyi et al., 2007). Should additional studies in TLR4^{+/-} infants confirm the inventors' observations, preventive interventions aimed at virus neutralization and/or novel anti-Th2 approaches (Wenzel et al., 2013) may be needed to protect these children.

On the other hand, given that ˜94% of infants in the studied region have two major alleles for TLR4, and high LPS levels-which are typical of poor environments- increase severity of RSV bronchiolitis among those children (p=0.03) (Collins et al., 2001 and Simoes, 2003), these findings may contribute to explain why poverty associates with severe RSV LRI (Nair et al., 2010). After all, treatment for RSV disease is supportive. Hence, sophisticated medical interventions are unlikely to explain outcome inequalities between socioeconomic groups. In fact, risk factors like crowding, day care attendance, exposure to cigarette smoke, and numerous siblings may further contribute to disease severity by enhancing the LPS/environmental effect on TLR4.

The inventors' observations suggest that environmental factors—interacting with the TLR4 genotype—modulate PRR expression in the respiratory tract and can result in an exaggerated Th2 response during RSV infection associated with severe bronchiolitis. While TLR4 studies provide an interesting window into RSV pathogenesis, other genes and risk factors have also been noted to influence disease severity (Hull et al., 2000, Openshaw, 1995, McNamara et al., 2004, Webb et al., 2003 and Nair et al., 2010). In fact, GATA3/T-bet ratios and Th2 bias may be part of a downstream pathogenic pathway modified at various levels by different genes affecting pulmonary function (Prince et al., 2005 and Soutiere et al., 2004) and/or immune responses (Eisenbarth et al., 2002) in the context of the environment.

These results have caveats. First, in studying human populations the inventors recognize that other environmental factors or unmeasured confounders may affect the results. Second, even though the inventors examined highly representative inflammatory cytokines, other unmeasured inflammatory molecules or even RSV load at other time points could still influence disease severity. In addition, studying healthy human subjects requires extrapolation of immune manifestations in the lungs through the analysis of upper respiratory tract secretions. The direct correlation of cytokine responses and virus titer during LRI has been described in other publications. Finally, although the LAL assay is the industry standard for assessment of endotoxicity, certain endotoxins may have different reactivity to the test.

However, this study has important strengths: first, it presents hundreds of infants from different environments with mild and severe RSV bronchiolitis and relevant RSV-negative controls in various populations; second, phenotypes in infants and in mice were defined by clinically relevant criteria; third, it identifies a novel population at high risk for severe disease, urban middle class TLR4+/− infants; and finally, it is the first study to propose a plausible mechanistic paradigm for environmental, genetic, epidemiological, viral, and immune factors affecting the pathogenesis of RSV bronchiolitis in infants and, simultaneously, offer mechanistic support for its observations in mice.

In summary, the inventors have described an association between TLR4 genotype and environmental conditions that effect through a Th2 transcription bias to modulate RSV disease severity. High IL-4/IFNγ ratios are associated with severe RSV bronchiolitis. Therapeutic interventions should explore modulating these molecules.

All of the methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference.
Alderborn et al., Genome Research 10(8):1249-1258 (2000).
Arbour et al., Nat Genet 25, 187-191 (2000).
Ausubel et al., CURRENT PROTOCOLS IN MOLECULAR BIOLOGY, John Wiley & Sons, N.Y. (2003).
Awomoyi et al., J Immunol 179, 3171-3177 (2007).
Braun-Fahrlander et al., N Engl J Med 347, 869-877 (2002).
Chen et al., Genome Research 9(5):492-498 (1999).
Church and Gilbert, Proc. Natl. Acad. Sci. USA 81:1991-1995 (1988).
Collins et al., FIELDS VIROLOGY 1443-1486 (2001).
Corneli et al., N Engl J Med 357, 331-339 (2007).
Cotton et al., Proc. Natl. Acad. Sci. USA 85:4397-4401 (1985).
Delgado et al., Nat Med 15, 34-41 (2009).
Eckert et al., PCR METHODS AND APPLICATIONS 1:17 (1991).
Ehl et al., Eur J Immunol 34, 1146-1153 (2004).
Eisenbarth et al., J Exp Med 196, 1645-1651 (2002).
Ferolla et al., Am J Respir Crit Care Med 187, 983-990 (2013).
Ferwerda et al., Proc. Natl. Acad. Sci. USA 104, 16645-16650 (2007).
Flavell et al., Cell 15:25 (1978).
Geever et al., Proc. Natl. Acad. Sci. USA 78:5081 (1981).
Glimcher, Nat Immunol 8, 448-450 (2007).
Guatelli et al., Proc. Nat. Acad. Sci. USA 87:1874 (1990).
Hall et al., N Engl J Med 360, 588-598 (2009).
Haynes et al., J Virol 75, 10730-10737 (2001).
Hoebee et al., J Infect Dis 187, 2-11 (2003).
Hoffman et al., Microbes Infect 6, 767-772 (2004).
Hull et al., Thorax 55, 1023-1027 (2000).
Inoue et al., J Infect Dis 195, 1618-1624 (2007).
Kaplan et al., Immunity 4, 313-319 (1996).
Karron et al., J Infect Dis 191, 1093-1104 (2005).
Kresfelder et al., J Med Virol 83, 1834-1840 (2011).
Kurt-Jones et al., Nat Immunol 1, 398-401 (2000).
Kwoh et al., Proc. Natl. Acad. Sci. USA 86:1173 (1989).
Laham et al., J Infect Dis 189, 2047-2056 (2004).
Landegren et al., Science 241:1077 (1988).
Larsson et al., Tob Induc Dis 4, 4 (2008).
Legg et al., Am J Respir Crit Care Med 168, 633-639 (2003).
Lukacs et al., Am J Pathol 169, 977-986 (2006).
Maniatis et al., Proc. Natl. Acad. Sci. USA 99:2228-2233 (2002).
Marr and Turvey, Innate Immun 18, 856-865 (2012).
Martinez, Proc Am Thorac Soc 4, 26-31 (2007).
Mattila et al., Nucleic Acids Res. 19:4967 (1991).
McNamara et al., Lancet 363, 1031-1037 (2004).
McPherson et al., PCR BASICS: FROM BACKGROUND TO BENCH, Springer-Verlag, (2000).
Miller, Nat Rev Microbiol 3, 36-46 (2005).
Morton et al., Proc. Natl. Acad. Sci. USA 98(9):5217-21 (2001).
Myers et al., Science 230:1242 (1985).
Nair et al., Lancet 375, 1545-1555 (2010).
Nath and Johnson, Biotechnic. Histochem. 73(1):6-22 (1998).
Nielsen et al., BIOCONJUGATE CHEMISTRY, The American Chemical Society, 5:1 (1994).
Openshaw, Am J Respir Crit Care Med 152, S59-62 (1995).
Orita et al., Proc. Natl. Acad. Sci. USA 86:2766-2770 (1989).
Palivizumab, Pediatrics 102, 531-537 (1998).
Paulus et al., Clin Immunol 123, 252-257 (2007).
PCR (eds. McPherson et al., IRL Press, Oxford).
PCR Primer: A Laboratory Manual, Dieffenbach and Dveksler, (Eds.).
Perros, et al., Respir Res 12, 125 (2011).
Prince et al., Dev Dyn 233, 553-561 (2005).
Prince et al., Genome Res. 11:152-162 (2001).
Puthothu et al., Dis Markers 22, 303-308 (2006).
Raca et al., Genet Test 8(4):387-94 (2004).
Robinson and O'Garra, Immunity 16, 755-758 (2002).
Saiki et al., Nature 324:163-166 (1986).
Sanger et al., Proc. Natl. Acad. Sci. USA 74:5463-5467 (1977).
Schafer et al., Nat. Biotechnol. 15:33-39 (1995).
Sheeran et al., Pediatr Infect Dis J 18, 115-122 (1999).
Sheffield et al., Proc. Natl. Acad. Sci. USA 86:232-236 (1989).
Simoes, J Pediatr 143, S118-126 (2003).
Simpson et al., Am J Respir Crit Care Med, 386-392 (2006).
Smit et al., Am J Respir Crit Care Med 179, 363-368 (2009).
Soutiere et al., Respir Physiol Neurobiol 140, 283-291 (2004).
Stoneking et al., Am. J. Hum. Genet. 48:370-382 (1991).
Tal et al., J Infect Dis 189, 2057-2063 (2004).
Tapper et al., Proc. Natl. Acad. Sci. USA 102(33):11835-11839 (2005).
The International HapMap Consortium, Nature 426:789-796 (2003).
The International HapMap Consortium, Nature 437:1299-1320 (2005).
U.S. Pat. No. 4,683,195
U.S. Pat. No. 4,683,202
U.S. Pat. No. 5,288,644
U.S. Pat. No. 5,491,224
U.S. Pat. No. 5,776,688
U.S. Pat. No. 5,800,998
U.S. Patent Publication No. 2004/0014095
Underhill et al., Genome Res. 7:996-1005 (1997).
Walsh et al., Science 320(5875):539-43 (2008).
Webb et al., Eur J Immunol 33, 3377-3385 (2003).
Welliver et al., J Infect Dis 195, 1126-1136 (2007).
Wenzel et al., N Engl J Med 368, 2455-2466 (2013).
Wheeless et al., Cytometry 17:319-326 (1994).
WO 99/57318
Wright, P. F. et al., J Infect Dis 185, 1011-1018 (2002).
Wu and Wallace, Genomics 4:560 (1989).
Zhu et al., Cell Res 16, 3-10 (2006).

What is claimed is:

1. A method of prophylactically treating a subject for respiratory syncytial virus (RSV) to reduce the chance of infection comprising:
(a) obtaining a genomic sample from a infant human subject;
(b) determining the presence or absence of the single nucleotide polymorphism (SNP) designated rs4986790 and/or rs4986791;
(c) determining lipopolysaccharide (LPS) exposure of said subject, and
(d) treating the subject prophylactically if said subject is heterozygous for the SNP designated rs4986790 and/or rs4986791, and LPS exposure is low.

2. The method of claim 1, wherein said subject is 1 to 61 days old.

3. The method of claim 1, wherein said subject is 1 to 12 months old.

4. The method of claim 1, wherein said sample is blood, a buccal cell sample, a nasal aspirate, or urine.

5. The method of claim 1, wherein treating comprises administering an anti-RSV antibody to said subject.

6. The method of claim 1, wherein treating comprises administering an anti-IL-4 antibody to said subject.

7. The method of claim 1, wherein treating comprises administering a pro-IFNγ agent to said subject.

8. The method of claim 1, wherein treating comprises administering low LPS levels to said subject.

9. The method of claim 1, further comprising treating said subject exhibiting either SNP with albuterol and/or corticosteroids in the event of an acute episode of bronchiolitis.

10. The method of claim 5, wherein said antibody is palivizumab.

11. The method of claim 1, wherein determining environmental LPS comprises (a) performing an LPS assay on a sample from said subject's home, or (b) performing a patient questionnaire that determines socio-economic status.

12. The method of claim 1, wherein determining environmental LPS is determined by immunoassay, mass spectrometry, gas chromatography, or biosassay.

13. The method of claim 8, wherein said bioassay is a limulus amebocyte assay.

14. The method of claim 1, wherein determining the presence of said SNP comprises sequencing, RFLP analysis, or primer extension.


SEQUENCE LISTING (7 sequences)

SEQ ID NO: 1 - DNA, 23 bp, Artificial sequence
  Synthetic RSV Taqman probe
caatgatcatgatttacctattg

SEQ ID NO: 2 - DNA, 19 bp, Artificial sequence
  Synthetic primer
atcatccttttcccacacc

SEQ ID NO: 3 - DNA, 18 bp, Artificial sequence
  Synthetic primer
aactcttcggctgcctct

SEQ ID NO: 4 - DNA, 24 bp, Artificial sequence
  Synthetic primer
ggaagggggaatttttctttaggc

SEQ ID NO: 5 - DNA, 21 bp, Artificial sequence
  Synthetic primer
ggcagtgtcctgatgactcag

SEQ ID NO: 6 - DNA, 20 bp, Artificial sequence
  Synthetic primer
taaacttgggagaacatggt

SEQ ID NO: 7 - DNA, 20 bp, Artificial sequence
  Synthetic primer
tggggaaagatagagtaata